(19) United States
(12) Patent Application Publication
(10) Pub. No.: US 2011/0306798 A1
(43) Pub. Date: Dec. 15, 2011

(54) Methods for Producing Arylsulfur Pentafluorides

(75) Inventor: Teruo Umemoto, Westminster, CO (US)

(73) Assignee: UBE INDUSTRIES, LTD., Ube-shi, Yamaguchi (JP)

(21) Appl. No.: 13/055,902

(22) PCT Filed: Jul. 29, 2009
(86) PCT No.: PCT/US09/52047

§ 371 Date: Mar. 21, 2011

Related U.S. Application Data
(60) Provisional application No. 61/084,780, filed on Jul. 30, 2008.

(51) Int. Cl. C07C 381/00

(52) U.S. Cl. 568/74

(57) ABSTRACT

Novel methods for preparing arylsulfur pentafluorides are disclosed. Arylsulfur halotetrafluoride is reacted with a fluoride source under hydrous conditions to form an arylsulfur pentafluoride. The purification method is also disclosed.

### TECHNICAL FIELD

The invention relates to methods useful in the preparation of arylsulfur pentafluorides.

### BACKGROUND OF THE INVENTION

Arylsulfur pentafluorides compounds are used to introduce one or more sulfur pentafluoride groups into various commercial organic molecules. In particular, arylsulfur pentafluorides have been shown as useful compounds (as product or intermediate) in the development of liquid crystals, in bioactive chemicals such as fungicides, herbicides, and insecticides, and in other like materials [see Fluorine-containing Synthons (ACS Symposium Series 911), ed by V. A. Soloshonok, American Chemical Society (2005), pp. 108-113]. However, as discussed herein, conventional synthetic methodologies to produce arylsulfur pentafluorides have proven difficult and are a concern within the art.

Generally, arylsulfur pentafluorides are synthesized using one of the following synthetic methods: (1) fluorination of diaryl disulfies or arylsulfur trifluoride with AgF₂ [see J. Am. Chem. Soc., Vol. 84 (1962), pp. 3064-3072, and J. Fluorine Chem. Vol. 112 (2001), pp. 287-295]; (2) fluorination of di(nitrophenyl)disulfides, nitrobenzenethiols, or nitrophenylsulfur trifluorides with molecular fluorine (F₂) [see Tetrahedron, Vol. 56 (2000), pp. 3399-3408; Eur. J. Org. Chem., Vol. 2005, pp. 3095-3100; and U.S. Pat. No. 5,741,935]; (3) fluorination of diaryl disulfides or arenethiols with F₂, CF₃OF, or CF₂(OF)₂ in the presence or absence of a fluoride source (see US Patent Publication No. 2004/0249209 A1); (4) fluorination of diaryl disulfides with XeF₂ [see J. Fluorine Chem., Vol. 101 (2000), pp. 279-283]; (5) reaction of 1,4-bis(acetoxy)-2-cyclohexene with SF₅Br followed by dehydrobromination or hydrolysis and then aromatization reactions [see J. Fluorine Chem., Vol. 125 (2004), pp. 549-552]; (6) reaction of 4,5-dichloro-1-cyclohexene with SF₅Cl followed by dehydrochlorination [see Organic Letters, Vol. 6 (2004), pp. 2417-2419 and PCT WO 2004/011422 A1]; and (7) reaction of SF₅Cl with acetylene, followed by bromination, dehydrobromination, and reduction with zinc, giving pentafluorosulfanylacetylene, which was then reacted with butadiene, followed by an aromatization reaction at very high temperature [see J. Org. Chem., Vol. 29 (1964), pp. 3567-3570].

Each of the above synthetic methods has one or more drawbacks making it either impractical (time or yield), overly expensive, and/or overly dangerous to practice. For example, synthesis methods (1) and (4) provide low yields and require expensive reaction agents, e.g., AgF₂ and XeF₂. Methods (2) and (3) require the use of F₂, CF₃OF, or CF₂(OF)₂, each of which is a toxic, explosive, and corrosive gas, and products produced using these methods are at a relatively low yield. Note that handling of these gasses is expensive from the standpoint of the gasses production, storage and use. In addition, synthesis methods that require the use of F₂, CF₃OF, and/or CF₂(OF)₂ are limited to the production of deactivated arylsulfur pentafluorides, such as nitrophenylsulfur pentafluorides, due to their extreme reactivity, which leads to side-reactions such as fluorination of the aromatic rings when not deactivated. Methods (5) and (6) also require expensive reactants, e.g., SF₅Cl and SF₅Br, and have narrow application because the starting cyclohexene derivatives are limited. Finally, method (7) requires the expensive reactant SF₅Cl and includes many reaction steps to reach the arylsulfur pentafluorides (timely and low yield). Therefore, problems with the production methods for arylsulfur pentafluorides have made it difficult to prepare the material in a safe, cost effective and timely fashion.

The present invention is directed toward overcoming one or more of the problems discussed above.

### SUMMARY OF THE INVENTION

The present invention provides useful methods for the production of arylsulfur pentafluoride, as represented by formula (I):

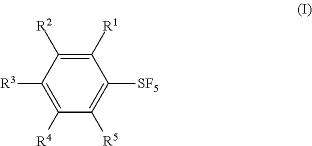

by reacting an arylsulfur halotetrafluoride having a formula (II) with a fluoride source under “hydrous conditions”. For purposes herein, hydrous conditions refer to reaction conditions that are performed in the presence of enough water molecules to no longer be considered non-hydrous or alternatively a non-limiting amount of water for the reaction to proceed as described herein.

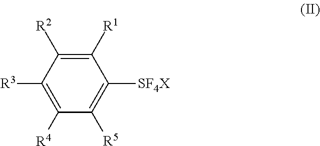

The present invention also provides a useful method for the production of arylsulfur pentafluoride, as represented by formula (I):

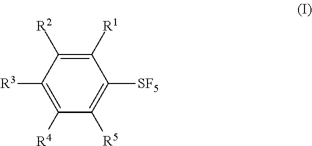

by reacting an arylsulfur halotetrafluoride having a formula (II) with a fluoride source under hydrous conditions;

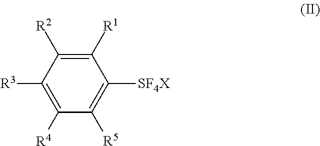

and treating the resulting products with hydrolysis conditions.

Embodiments of the present invention also provide a method for producing an arylsulfur pentafluoride (formula I) by reacting an arylsulfur halotetrafluoride having a formula (II) with a fluoride source under hydrous conditions in the presence of a halogen, the halogen selected from the group of chlorine, bromine, iodine, and interhalogens.

Embodiments of the present invention also provide a method for producing an arylsulfur pentafluoride (formula I) by reacting an arylsulfur halotetrafluoride having a formula (II) with a fluoride source under hydrous conditions in the presence of a halogen, the halogen selected from the group of chlorine, bromine, iodine, and interhalogens, and treating the resulting products with hydrolysis conditions. Hydrolysis conditions refer to reaction conditions under which hydrolysis of a byproduct(s) takes place.

In addition, embodiments of the present invention provide a purification method for an arylsulfur pentafluoride (formula I) by treating a mixture containing the arylsulfur pentafluoride with hydrolysis conditions. The method includes hydrolysis in the presence or absence of a base or an acid with or without a phase-transfer catalyst.

These and various other features as well as advantages which characterize embodiments of the invention will be apparent from a reading of the following detailed description and a review of the appended claims.

### DETAILED DESCRIPTION OF THE INVENTION

Embodiments of the present invention provide industrially useful methods for producing arylsulfur pentafluorides, as represented by formula (I). Prepared arylsulfur pentafluorides can be used, for among other things, to introduce one or more sulfur pentafluoride (SF₅) groups into various target organic compounds. Target organic compounds herein include a wide range of organic compounds that may have potential use in application when one or more SF₅ groups are introduced as a part of molecule(s).

Unexpectedly, we found that water molecules accelerate the reaction of an arylsulfur halotetrafluoride of formula (II) with a fluoride source, giving an arylsulfur pentafluoride of formula (I) (see Example 17 and Comparative Example 1, Example 18 and Comparative Example 2, and Example 21 and Comparative Example 3). In addition, unlike previous methods in the art, the methods of the invention can utilize low cost reagents such as “hydrous” fluoride sources and starting materials (arylsulfur halotetrafluorides) that can be produced at low cost.

Many fluoride sources are prepared from aqueous solution using hydrofluoric acid. Fluoride sources that are active in the reactions of this invention generally have a strong affinity with water due to strong hydrogen bonding formation or Lewis or Bronsted acidic nature. Anhydrous fluoride sources become easily hydrous on handling or during long storage because fluoride sources may be hygroscopic. Note that it is not typically easy to completely remove water from a fluoride source. For example, heating at high temperature at reduced pressure for long periods of time is needed. In some cases, because heating makes the fluoride source decomposed, a costly method is needed for the preparation of an anhydrous fluoride source. Accordingly, it can be costly to make an anhydrous fluoride source. The present invention, however, can use hydrous fluoride sources and avoid the cost and enhanced technical difficulty of preparing anhydrous fluoride sources.

The starting materials, arylsulfur halotetrafluorides, can be prepared by reactions of diaryl disulfides or arylthiols with a halogen gas such as chlorine (Cl₂) and a fluoro salt such as metal fluoride under mild reaction conditions, as mentioned in details below. The diaryl disulfides and arylthiols are of low cost and easily prepared, and chlorine gas and meal fluorides are also of low cost. Therefore, the arylsulfur halotetrafluorides can be prepared in a large scale for a low cost.

Further, methods of the invention provide a greater degree of safety in comparison to most prior art methodologies (see for example methods that use F₂ gas). A distinction of the present invention is that the methods herein afford low cost and high safety, as compared to other conventional methods.

Table 1 provides structure names and formulas for reference when reviewing Schemes 1-8 and Examples 1-27:

TABLE 1
Formulas (I, II, IIIa, IIIb, IV, V, VI, and VII)

| Name | Structure/Formula Number |
|---|---|
| Arylsulfur pentafluoride | 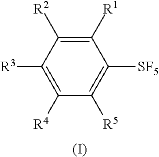 |
| Arylsulfur halotetrafluoride | 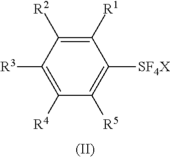 |
| Aryl sulfur compound | 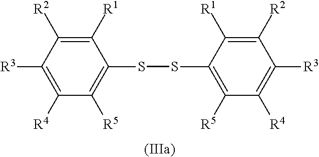 |
| Aryl sulfur compound | 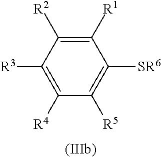 |
| Fluoro salt | M⁺F⁻ (IV) |
| Arylsulfur trifluoride | 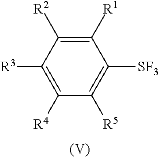 |
| Arylsulfonyl fluoride | 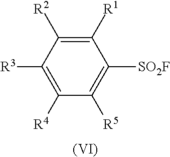 |
| Arylsulfonic acid or salt | 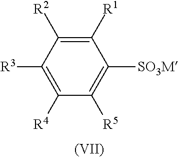 |

Embodiments of the invention include a method (Scheme I; process I) for preparing an arylsulfur pentafluoride having a formula (I), which comprises reacting an arylsulfur halotetrafluoride having a formula (II) with a fluoride source under hydrous conditions. For purposes herein hydrous conditions indicate the presence of some level of H₂O in the reaction.

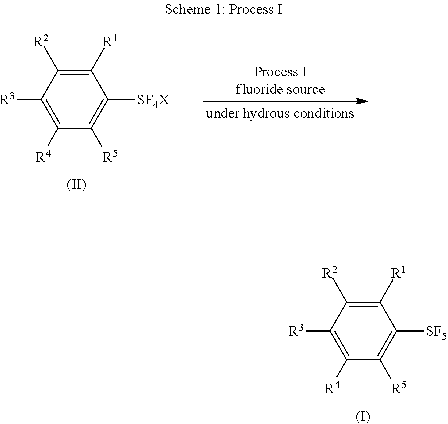

For the compounds represented by formulas (I) and (II), R¹, R², R³, R⁴, and R⁵ each is independently a hydrogen atom; a halogen atom that is a fluorine atom, a chlorine atom, a bromine atom, or an iodine atom; a substituted or unsubstituted alkyl group having from 1 to 18 carbon atoms, preferably from 1 to 10 carbon atoms; a substituted or unsubstituted aryl group having from 6 to 30 carbon atoms, preferably from 6 to 15 carbon atoms; a nitro group; a cyano group; a substituted or unsubstituted alkanesulfonyl group having from 1 to 18 carbon atoms, preferably from 1 to 10 carbon atoms; a substituted or unsubstituted arenesulfonyl group having from 6 to 30 carbon atoms, preferably from 6 to 15 carbon atoms; a substituted or unsubstituted alkoxy group having from 1 to 18 carbon atoms, preferably from 1 to 10 carbon atoms; a substituted or unsubstituted aryloxy group having from 6 to 30 carbon atoms, preferably from 6 to 15 carbon atoms; a substituted or unsubstituted acyloxy group having from 1 to 18 carbon atom, preferably from 1 to 10 carbon atoms; a substituted or unsubstituted alkanesulfonyloxy group having from 1 to 18 carbon atom, preferably from 1 to 10 carbon atoms; a substituted or unsubstituted arenesulfonyloxy group having from 6 to 30 carbon atoms, preferably from 6 to 15 carbon atoms; a substituted or unsubstituted alkoxycarbonyl group having 2 to 18 carbon atoms, preferably from 2 to 10 carbon atoms; a substituted or unsubstituted aryloxycarbonyl group having 7 to 30 carbon atoms, preferably from 7 to 15 carbon atoms; a substituted carbamoyl group having 2 to 18 carbon atoms, preferably from 2 to 10 carbon atoms; a substituted amino group having 1 to 18 carbon atoms, preferably from 1 to 10 carbon atoms; or a SF₅ group. With regard to X, X is a chlorine atom, a bromine atom, or an iodine atom.

The term “alkyl” as used herein is linear, branched, or cyclic alkyl. The alkyl part of alkanesulfonyl, alkoxy, alkanesulfonyloxy, or alkoxycarbonyl group as used herein is also linear, branched, or cyclic alkyl part. When an acyloxy group contains an alkyl part, the alkyl part is also linear, branched, or cyclic alkyl part.

The term “substituted alkyl” as used herein means an alkyl moiety having one or more substituents such as a halogen atom, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted aryl” as used herein means an aryl moiety having one or more substituents such as a halogen atom, a substituted or unsubstituted alkyl group, and/or any other group with or without a heteroatom(s) such as an oxygen atom(s), a nitrogen atom(s), and/or a sulfur atom(s), which does not limit reactions of this invention.

The term “substituted alkanesulfonyl” as used herein means an alkanesulfonyl moiety having one or more substituents such as a halogen atom, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted arenesulfonyl” as used herein means an arenesulfonyl moiety having one or more substituents such as a halogen atom, a substituted or unsubstituted alkyl group, and/or any other group with or without a heteroatom(s) such as an oxygen atom(s), a nitrogen atom(s), and/or a sulfur atom(s), which does not limit reactions of this invention.

The term “substituted alkoxy” as used herein means an alkoxy moiety having one or more substituents such as a halogen atom, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted aryloxy” as used herein means an aryloxy moiety having one or more substituents such as a halogen atom, a substituted or unsubstituted alkyl group, and/or any other group with or without a heteroatom(s) such as an oxygen atom(s), a nitrogen atom(s), and/or a sulfur atom(s), which does not limit reactions of this invention.

The term “substituted acyloxy” as used herein means an acyloxy moiety having one or more substituents such as a halogen atom, a substituted or unsubstituted alkyl group, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted alkanesulfonyloxy” as used herein means an alkanesulfonyloxy moiety having one or more substituents such as a halogen atom, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted arenesulfonyloxy” as used herein means an arenesulfonyloxy moiety having one or more substituents such as a halogen atom, a substituted or unsubstituted alkyl group, and/or any other group with or without a heteroatom(s) such as an oxygen atom(s), a nitrogen atom(s), and/or a sulfur atom(s), which does not limit reactions of this invention.

The term “substituted alkoxycarbonyl” as used herein means an alkoxycarbonyl moiety having one or more substituents such as a halogen atom, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted aryloxycarbonyl” as used herein means an aryloxycarbonyl moiety having one or more substituents such as a halogen atom, a substituted or unsubstituted alkyl group, and/or any other group with or without a heteroatom(s) such as an oxygen atom(s), a nitrogen atom(s), and/or a sulfur atom(s), which does not limit reactions of this invention.

The term “substituted carbamoyl” as used herein means a carbamoyl moiety having one or more substituents such as a substituted or unsubstituted alkyl group, a substituted or unsubstituted aryl group, and/or any other group with or without a heteroatom(s) such as an oxygen atom(s), a nitrogen atom(s), and/or a sulfur atom(s), which does not limit reactions of this invention.

The term “substituted amino” as used herein means an amino moiety having one or more substituents such as a substituted or unsubstituted acyl group, a substituted or unsubstituted alkanesulfonyl group, a substituted or unsubstituted arenesulfonyl group, and/or any other group with or without a heteroatom(s) such as an oxygen atom(s), a nitrogen atom(s), and/or a sulfur atom(s), which does not limit reactions of this invention.

Among R¹, R², R³, R⁴, and R⁵, described above, a hydrogen atom, a halogen atom, a substituted or unsubstituted alkyl group, a substituted or unsubstituted aryl group, a nitro group, a cyano group, a substituted or unsubstituted alkanesulfonyl group, a substituted or unsubstituted arenesulfonyl group, a substituted or unsubstituted alkoxy group, a substituted or unsubstituted aryloxy group, a substituted or unsubstituted acyloxy group, and a substituted or unsubstituted alkoxycarbonyl group are preferable, and a hydrogen atom, a halogen atom, a substituted or unsubstituted alkyl group, a substituted or unsubstituted aryl group, and a nitro group are more preferable from the viewpoint of availability of the starting materials.

Note that according to the nomenclature of Chemical Abstract Index Name, and in accordance with the present disclosure, for example, C₆H₅—SF₅ is named sulfur, pentafluorophenyl-; p-Cl—C₆H₄—SF₅ is named sulfur, (4-chlorophenyl)pentafluoro-; and p-CH₃—C₆H₄—SF₅ is named sulfur, pentafluoro(4-methylphenyl)-. C₆H₅—SF₄Cl is named sulfur, chlorotetrafluorophenyl-; p-CH₃—C₆H₄—SF₄Cl is named sulfur, chlorotetrafluoro(4-methylphenyl)-; and p-NO₂—C₆H₄—SF₄Cl is named sulfur, chlorotetrafluoro(4-nitrophenyl)-.

Arylsulfur halotetrafluoride compounds of formula (II) include isomers such as trans-isomers and cis-isomers as shown below; arylsulfur halotetrafluoride is represented by ArSF₄X:

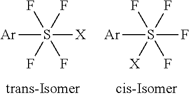

One embodiment of the invention includes a method that under hydrous conditions, an arylsulfur halotetrafluoride is reacted with a fluoride source. The hydrous conditions can be produced, for example, by: (1) a hydrous or wet fluoride source; (2) a wet or aqueous solvent; (3) a wet arylsulfur halotetrafluoride; (4) water, steam, or water vapor added to a fluoride source, an arylsulfur halotetrafluoride, solvent, and/or a reaction mixture or a reaction system; (5) humid air introduced to a fluoride source, an arylsulfur halotetrafluoride, solvent, and/or a reaction mixture or a reaction system; and/or (6) the reaction is run in a humid or moist conditions, i.e., non-anhydrous conditions.

The starting materials, arylsulfur halotetrafluorides, used for this invention may be prepared according to the method shown in the literature (see Can. J. Chem., Vol. 75, pp. 1878-1884). The arylsulfur halotetrafluorides can be prepared industrially at low cost in the following ways (Process A or B);

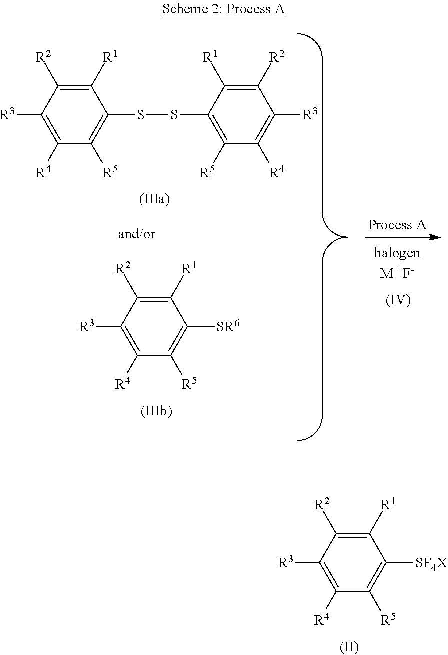

Process A includes reacting at least one aryl sulfur compound, having a formula (IIIa) or (IIIb), with a halogen selected from the group of chlorine, bromine, iodine and interhalogens, and a fluoro salt (M⁺F, formula IV) to form an arylsulfur halotetrafluoride having a formula (II). R¹, R², R³, R⁴, R⁵, and X are described as above. R⁶ is a hydrogen atom, a silyl group, a metal atom, an ammonium moiety, a phosphonium moiety, or a halogen atom.

Illustrative aryl sulfur compounds, as represented by formula (IIIa), used in Process A include, but are not limited to: diphenyl disulfide, each isomer of bis(fluorophenyl)disulfide, each isomer of bis(difluorophenyl)disulfide, each isomer of bis(trifluorophenyl)disulfide, each isomer of bis(tetrafluorophenyl)disulfide, bis(pentafluorophenyl)disulfide, each isomer of bis(chlorophenyl)disulfide, each isomer of bis(dichorophenyl)disulfide, each isomer of bis(trichlorophenyl)disulfide, each isomer of bis(bromophenyl)disulfide, each isomer of bis(dibromophenyl)disulfide, each isomer of bis(iodophenyl)disulfide, each isomer of bis(chlorofluorophenyl)disulfide, each isomer of bis(bromofluorophenyl)disulfide, each isomer of bis(bromochlorophenyl)disulfide, each isomer of bis(fluoroiodophenyl)disulfide, each isomer of bis(tolyl)disulfide, each isomer of bis[(methoxymethyl)phenyl]disulfide, each isomer of bis{[(cyclohexyloxy)methyl]phenyl}disulfide, each isomer of bis[(phenylmethyl)phenyl]disulfide, each isomer of bis[(cyanomethyl)phenyl]disulfide, each isomer of bis[(nitromethyl)phenyl]disulfide, each isomer of bis{[(methanesulfonyl)methyl]phenyl}disulfide, each isomer of bis{[(benzenesulfonyl)methyl]phenyl}disulfide, each isomer of bis(ethylphenyl)disulfide, each isomer of bis[(methoxyethyl)phenyl]disulfide, each isomer of bis[(nitroethyl)phenyl]& disulfide, each isomer of bis[(phenylethyl)phenyl]disulfide, each isomer of bis[chloro(methyl)phenyl]disulfide, each isomer of bis[bromo(methyl)phenyl]disulfide, each isomer of bis[(trifluoromethyl)phenyl]disulfide, each isomer of bis(dimethylphenyl)disulfide, each isomer of bis[chloro(dimethyl)phenyl]disulfide, each isomer of bis[di(trifluoromethyl)phenyl]disulfide, each isomer of bis(trimethylphenyl)disulfide, each isomer of bis[chloro(trimethyl)phenyl]disulfide, each isomer of bis(tetramethylphenyl)disulfide, each isomer of bis[chloro(tetramethyl)phenyl]disulfide, bis(pentamethylphenyl)disulfide, each isomer of bis(ethylphenyl)disulfide, each isomer of bis{(2,2,2-trifluoroethyl)phenyl]disulfide, each isomer of bis[(perfluoroethyl)phenyl]disulfide, each isomer of bis(diethylphenyl)disulfide, each isomer of bis[ethyl(methyl)phenyl]disulfide, each isomer of bis(propylphenyl)disulfide, each isomer of bis(isopropylphenyl)disulfide, each isomer of bis(butylphenyl)disulfide, each isomer of bis(sec-butylphenyl)disulfide, each isomer of bis(isobutylphenyl)disulfide, each isomer of bis(tert-butylphenyl)disulfide, each isomer of bis(cyclopropylphenyl)disulfide, each isomer of bis(cyclopentylphenyl)disulfide, each isomer of bis(cyclohexylphenyl)disulfide, each isomer of bis{[(cyclohexyl)cyclohexyl]phenyl}disulfide, each isomer of bis(biphenyl)disulfide, each isomer of bis(tolylphenyl)disulfide, each isomer of bis[(chlorophenyl)phenyl]disulfide, each isomer of bis[(bromophenyl)phenyl]disulfide, each isomer of bis[(nitrophenyl)phenyl]disulfide, each isomer of bis(terphenylyl)disulfide, each isomer of bis[(phenyl)terphenylyl]disulfide, each isomer of bis(nitrophenyl)disulfide, each isomer of bis(dinitrophenyl)disulfide, each isomer of bis(cyanophenyl)disulfide, each isomer of bis(dicyanophenyl)disulfide, each isomer of bis[(methanesulfonyl)phenyl]disulfide, each isomer of bis[(trifluoromethanesulfonyl)phenyl]disulfide, each isomer of bis[(benzenesulfonyl)phenyl]disulfide, each isomer of bis[(toluenesulfonyl)phenyl]disulfide, each isomer of bis(methoxyphenyl)disulfide, each isomer of bis(ethoxyphenyl)disulfide, each isomer of bis(propoxyphenyl)disulfide, each isomer of bis(butoxyphenyl)disulfide, each isomer of bis(cyclopentyloxyphenyl)disulfide, bis(cyclohexyloxyphenyl)disulfide, each isomer of bis[(trifluoromethoxy)phenyl]disulfide, each isomer of bis[(perfluoroethoxyl)phenyl]disulfide, each isomer of bis[(trifluoroethoxy)phenyl]disulfide, each isomer of bis[(tetrafluoroethoxy)phenyl]disulfide, each isomer of bis[(perfluoropropoxy)phenyl]disulfide, each isomer of bis(phenyloxyphenyl)disulfide, each isomer of bis(fluorophenyloxyphenyl)disulfide, each isomer of bis(chlorophenyloxyphenyl)disulfide, each isomer of bis(bromophenyloxyphenyl)disulfide, each isomer of bis(nitrophenyloxyphenyl)disulfide, each isomer of bis[(dinitrophenyloxy)phenyl]disulfide, each isomer of bis[(pentafluorophenyloxy)phenyl]disulfide, each isomer of bis(trifluoromethylphenyloxyphenyl)disulfide, each isomer of bis(cyanophenyloxyphenyl)disulfide, each isomer of bis(naphthyloxylphenyl)disulfide, each isomer of bis[(heptafluoronaphthyloxy)phenyl]disulfide, each isomer of bis(acetoxyphenyl)disulfide, each isomer of bis[(benzoyloxy)phenyl]disulfide, each isomer of bis[(methanesulfonyloxy)phenyl]disulfide, each isomer of bis[(benzenesulfonyloxy)phenyl]disulfide, each isomer of bis[(toluenesulfonyloxy)phenyl]disulfide, each isomer of bis[(methoxycarbonyl)phenyl]disulfide, each isomer of bis[(ethoxycarbonyl)phenyl]disulfide, each isomer of bis[(phenoxycarbonyl)phenyl]disulfide, each isomer of bis[(N,N-dimethylcarbamoyl)phenyl]disulfide, each isomer of bis[(N,N-diethylcarbamoyl)phenyl]disulfide, each isomer of bis[(N,N-diphenylcarbamoyl)phenyl]disulfide, each isomer of bis[(N,N-dibenzylcarbamoyl)phenyl]disulfide, each isomer of bis[(N-acetyl-N-methylamino)phenyl]disulfide, each isomer of bis[(N-acetyl-N-phenylamino)phenyl]disulfide, each isomer of bis[(N-acetyl-N-benzylamino)phenyl]disulfide, each isomer of bis[(N-benzoyl-N-methylamino)phenyl]disulfide, each isomer of bis[(N-methanesulfonyl-N-methylamino)phenyl]disulfide, each isomer of bis[(N-toluenesulfonyl-N-methylamino)phenyl]disulfide, each isomer of bis[(N-toluenesulfonyl-N-benzylamino)phenyl]disulfide, and each isomer of bis[(pentafluorosulfanyl)phenyl]disulfide. Each of the above formula (IIIa) compounds is available (see for example Sigma-Aldrich, Acros, TCI, Lancaster, Alfa Aesar, etc.) or can be prepared in accordance with understood principles of synthetic chemistry.

Illustrative aryl sulfur compounds, as represented by formula (IIIb), used in Process A include, but are not limited to: benzenethiol, each isomer of fluorobenzenethiol, each isomer of chlorobenzenethiol, each isomer of bromobenzenethiol, each isomer of iodobenzenethiol, each isomer of difluorobenzenethiol, each isomer of trifluorobenzenethiol, each isomer of tetrafluorobenzenethiol, pentafluorobenzenethiol, each isomer of dichlorobenzenethiol, each isomer of chlorofluorobenzenethiol, each isomer of methylbenzenethiol, each isomer of (trifluoromethyl)benzenethiol, each isomer of dimethylbenzenethiol, each isomer of bis(trifluoromethyl)benzenethiol, each isomer of methyl(trifluoromethyl)benzenethiol, each isomer of trimethylbenzenethiol, each isomer of tetramethylbenzenethiol, pentamethylbenzenethiol, each isomer of ethylbenzenethiol, each isomer of (2,2,2-trifluoroethyl)benzenethiol, each isomer of (perfluoroethyl)benzenethiol, each isomer of diethylbenzenethiol, each isomer of ethyl(methyl)benzenethiol, each isomer of propylbenzenethiol, each isomer of isopropylbenzenethiol, each isomer of butylbenzenethiol, each isomer of sec-butylbenzenethiol, each isomer of isobutylbenzenethiol, each isomer of tert-butylbenzenethiol, each isomer of nitrobenzenethiol, each isomer of dinitrobenzenethiol, each isomer of cyanobenzenethiol, each isomer of phenylbenzenethiol, each isomer of tolylbenzenethiol, each isomer of (chlorophenyl)benzenethiol, each isomer of (bromophenyl)benzenethiol, each isomer of (nitrophenyl)benzenethiol, each isomer of (methanesulfonyl)benzenethiol, each isomer of (trifluoromethanesulfonyl)benzenethiol, each isomer of (benzenesulfonyl)benzenethiol, each isomer of (toluenesulfonyl)benzenethiol, each isomer of (methoxycarbonyl)benzenethiol, each isomer of (ethoxycarbonyl)benzenethiol, each isomer of (phenoxycarbonyl)benzenethiol, each isomer of (N,N-dimethylcarbamoyl)benzenethiol, each isomer of (N,N-diethylcarbamoyl)benzenethiol, each isomer of (N,N-dibenzylcarbamoyl)benzenethiol, each isomer of (N,N-diphenylcarbamoyl)benzenethiol, each isomer of (N-acetyl-N-methylamino)benzenethiol, each isomer of (N-acetyl-N-phenylamino)benzenethiol, each isomer of (N-acetyl-N-benzylamino)benzenethiol, each isomer of (N-benzoyl-N-methylamino)benzenethiol, each isomer of (N-methanesulfonyl-N-methylamino)benzenethiol, each isomer of (N-toluenesulfonyl-N-methylamino)benzenethiol, each isomer of (N-toluenesulfonyl-N-benzylamino)benzenethiol, and each isomer of (pentafluorosulfanyl)benzenethiol; lithium, sodium, and potassium salts of the benzenethiol compounds exemplified here; ammonium, diethylammonium, triethylammonium, trimethylammnoim, tetramethylammonium, tetraethylammonium, tetrapropylammonium, and tetrabutylammonium salts of the benzenethiol compounds exemplified here; tetramethylphosphonium, tetraethylphosphonium, tetrapropylphosphonium, tetrabutylphosphonium, and tetraphenylphosphonium salts of the benzenethiol compounds exemplified here; and S-trimethylsilyl, S-triethylsilyl, S-tripropylsilyl, S-dimethyl-t-butylsilyl, and S-dimethylphenylsilyl derivative of the benzenethiol compounds exemplified here. Examples of aryl sulfur compounds of formula (IIIb) where R⁶ is a halogen atom are benzenesulfenyl chloride, each isomer of nitrobenzenesulfenyl chloride, each isomer of dinitrobenzenesulfenyl chloride, and other like compounds. Each of the above formula (IIIb) compounds is available from appropriate vendors (see for example Sigma-Aldrich, Acros, TCI, Lancaster, Alfa Aesar, etc.) or can be prepared in accordance with known principles of synthetic chemistry.

Typical halogens employable in Process A include chlorine (Cl₂), bromine (Br₂), iodine (I₂), and interhalogens such as ClF, BrF, ClBr, ClI, Cl₃I, and BrI. Among these, chlorine (Cl₂) is preferable due to its low cost.

Fluoro salts, having a formula (IV), are those which are easily available and are exemplified by metal fluorides, ammonium fluorides, and phosphonium fluorides. Examples of suitable metal fluorides are alkali metal fluorides such as lithium fluoride, sodium fluoride, potassium fluoride (including spray-dried potassium fluoride), rubidium fluoride, and cesium fluoride. Examples of suitable ammonium fluorides are tetramethylammonium fluoride, tetraethylammonium fluoride, tetrapropylammonium fluoride, tetrabutylammonium fluoride, benzyltrimethylammonium fluoride, benzyltriethylammonium fluoride, and so on. Examples of suitable phosphonium fluorides are tetramethylphosphonium fluoride, tetraethylphosphonium fluoride, tetrapropylphosphonium fluoride, tetrabutylphosphonium fluoride, tetraphenylphosphonium fluoride, tetratolylphosphonium fluoride, and so on. The alkali metal fluorides, such as potassium fluoride and cesium fluoride, are preferable from the viewpoint of availability and capacity to result in high yield, and potassium fluoride is most preferable from the viewpoint of cost.

From the viewpoint of efficiency and yields of the reactions, Process A is preferably carried out in the presence of one or more solvents. Preferable solvents include an inert, polar, or aprotic solvent. Preferable solvents will not substantially react with the starting materials and reagents, the intermediates, and/or the final products. Suitable solvents include, but are not limited to, nitriles, ethers, nitro compounds, and so on, and mixtures thereof. Illustrative nitriles are acetonitrile, propionitrile, benzonitrile, and so on. Illustrative ethers are tetrahydrofuran, diethyl ether, dipropyl ether, dibutyl ether, tert-butyl methyl ether, dioxane, glyme, diglyme, triglyme, and so on. Illustrative nitro compounds are nitromethane, nitroethane, nitropropane, nitrobenzene, and so on. Acetonitrile is a preferred solvent for use in Process A from a viewpoint of providing higher yields of the products.

In order to obtain good yields of product in Process A, the reaction temperature can be selected in the range of about −60° C.˜+70° C. More preferably, the reaction temperature can be selected in the range of about −40° C.˜+50° C. Furthermore preferably, the reaction temperature can be selected in the range of about −20° C.˜+40° C.

Reaction conditions of Process A are optimized to obtain economically good yields of product. In one illustrative embodiment, from about 5 mol to about 20 mol of halogen are combined with about 1 mol of aryl sulfur compound (formula Ma) to obtain a good yield of arylsulfur halotetrafluorides (formula II). In another embodiment, from about 3 to about 12 mol of halogen are combined with about 1 mol of aryl sulfur compound of formula IIIb (R⁶=a hydrogen atom, a silyl group, a metal atom, an ammonium moiety, or a phosphonium moiety) to obtain good yields of arylsulfur halotetrafluorides (formula II). From about 2 to about 8 mol of halogen are combined with about 1 mol of aryl sulfur compound of formula IIIb (R⁶=a halogen atom) to obtain good yields of arylsulfur halotetrafluorides (formula II).

The amount of a fluoro salt (formula IV) used in embodiments of Process A can be in the range of from about 8 to about 24 mol against about 1 mol of aryl sulfur compound of formula (IIIa) to obtain economically good yields of product. The amount of a fluoro salt (formula IV) used in embodiments of Process A can be in the range of from about 4 to about 12 mol against about 1 mol of aryl sulfur compound of formula (IIIb) to obtain economically good yields of product.

Note that the reaction time for Process A varies dependent upon reaction temperature, and the types and amounts of substrates, reagents, and solvents. As such, reaction time is generally determined as the amount of time required to complete a particular reaction, but can be from about 0.5 h to several days, preferably, within a few days.

In some cases, R¹, R², R³, R⁴, and/or R⁵ of the compounds represented by the formula (II) may be different from R¹, R², R³, R⁴, and/or R⁵ of the starting materials represented by the formulas (IIIa) and/or (IIIb). Transformation of the R¹, R², R³, R⁴, and/or R⁵ to different R¹, R², R³, R⁴, and/or R⁵ may take place under the reaction conditions of Process A.

The starting materials, arylsulfur halotetrafluorides having a formula (II), can be also prepared in the following way, see for example (Process B):

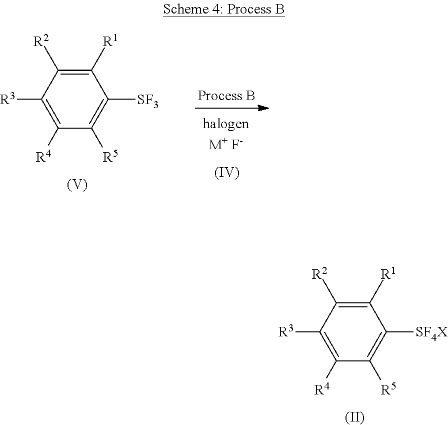

An arylsulfur halotetrafluoride having a formula (II) can be produced by reacting an arylsulfur trifluoride having a formula (V) with a halogen selected from the group of chlorine, bromine, iodine, and interhalogens and a fluoro salt (formula IV). For R¹, R², R³, R⁴, R⁵, and X of formulas (V) and (II), each has the same meaning as previously described.

Illustrative arylsulfur trifluorides, as represented by formula (V), used in Process B can be prepared as described in the literature [see J. Am. Chem. Soc., Vol. 84 (1962), pp. 3064-3072, and Synthetic Communications, Vol. 33 (2003), pp. 2505-2509 each of which is incorporated by reference for all purposes] and are exemplified, but are not limited, by phenylsulfur trifluoride, each isomer of fluorophenylsulfur trifluoride, each isomer of difluorophenylsulfur trifluoride, each isomer of trifluorophenylsulfur trifluoride, each isomer of tetrafluorophenylsulfur trifluoride, pentafluorophenylsulfur trifluoride, each isomer of chlorophenylsulfur trifluoride, each isomer of bromophenylsulfur trifluoride, each isomer of chlorofluorophenylsulfur trifluoride, each isomer of bromofluorophenylsulfur trifluoride, each isomer of tolylsulfur trifluoride, each isomer of chloro(methyl)phenylsulfur trifluoride, each isomer of dimethylphenylsulfur trifluoride, each isomer of chloro(dimethyl)phenylsulfur trifluoride, each isomer of trimethylphenylsulfur trifluoride, each isomer of ethylphenylsulfur trifluoride, each isomer of propylphenylsulfur trifluoride, each isomer of butylphenylsulfur trifluoride, each isomer of nitrophenylsulfur trifluoride, each isomer of dinitrophenylsulfur trifluoride, and so on.

Arylsulfur trifluorides (formula V) can be intermediates in Process A.

A halogen employable for Process B is the same as for Process A described above, except for the amount used in the reaction(s).

Fluoro salts having a formula (IV) for Process B are the same as for Process A described above, except for the amount used in the reaction(s).

It is preferable that the reaction of Process B be carried out using a solvent. Examples of suitable solvents for Process B are the same as for Process A described above.

In order to get good economic yields of the products, the reaction temperature for Process B can be selected in the range of −60° C.˜+70° C. More preferably, the temperature can be selected in the range of −40° C.˜+50° C. Furthermore preferably, the temperature can be selected in the range of −20° C.˜+40° C.

In order to get good economic yields of product, the amount of a halogen used can be preferably selected in the range of from about 1 to about 5 mol, more preferably from about 1 to about 3 mol, against about 1 mol of arylsulfur trifluoride (V).

In order to get good economic yield of the products, the amount of fluoro salt (IV) used can be preferably selected in the range of about 1 to about 5 mol against about 1 mol of arylsulfur trifluoride (V).

The reaction time for Process B is dependent on reaction temperature, the substrates, reagents, solvents, and their amounts used. Therefore, one can choose the time necessary for completing each reaction based on modification of the above parameters, but can be from about 0.5 h to several days, preferably, within a few days.

In some cases, R¹, R², R³, R⁴, and/or R⁵ of the compounds represented by the formula (II) may be different from R¹, R², R³, R⁴, and/or R⁵ of the starting materials represented by the formula (V). Transformation of the R¹, R², R³, R⁴, and/or R⁵ to different R¹, R², R³, R⁴, and/or R⁵ may take place under the reaction conditions of Process B.

Fluoride sources employable in Process I (Scheme 1) of this invention are compounds that display fluoride activity to the arylsulfur halotetrafluoride (formula II). The fluoride sources may be hydrous or anhydrous (assuming other aspects or the reaction are hydrous). The fluoride sources can be selected from fluorides of typical elements in the Periodic Table, fluorides of transition elements in the Periodic Table, and mixtures or compounds between or among these fluorides of typical elements and/or transition elements. The fluoride source may be a mixture, salt, or complex with an organic molecule(s) that does(do) not limit the reactions of this invention. The fluoride sources also include mixtures or compounds of fluoride sources with fluoride source-activating compounds such as SbCl₅, AlCl₃, PCl₅, BCl₃, and so on. Process I can be carried out using one or more fluoride sources.

Suitable examples of fluorides of the typical elements include fluorides of Element 1 in the Periodic Table such as hydrogen fluoride (HF) and alkali metal fluorides, LiF, NaF, KF, RbF, and CsF; fluorides of Element 2 (alkaline earth metal fluorides) such as BeF₂, MgF₂, MgFCl, CaF₂, SrF₂, BaF₂ and so on; fluorides of Element 13 such as BF₃, BF₂Cl, BFCl₂, AlF₃, AlF₂Cl, AlFCl₂, GaF₃, InF₃, and so on; fluorides of Element 14 such as SiF₃, SiF₃Cl, SiF₂Cl₂, SiFCl₃, GeF₄, GeF₂Cl₂, SnF₄, PbF₂, PbF₄, and so on; fluorides of Element 15 such as PF₃, PF₅, AsF₃, AsF₅, SbF₃, SbF₅, SbF₄Cl, SbF₃Cl₂, SbF₂Cl₃, SbFCl₄, BiF₅, and so on; fluorides of Element 16 such as OF₂, SeF₄, SeF₆, TeF₄, TeF₆, and so on; fluorides of Element 17 such as F₂, ClF, ClF₃, BrF, BrF₃, IF₅, and so on.

Suitable examples of fluorides of the transition elements (transition metal fluorides) include fluorides of Element 3 in the Periodic Table such as ScF₃, YF₃, LaF₃, and so on; fluorides of Element 4 such as TiF₄, ZrF₃, ZrF₄, HfF₄, and so on; fluorides of Element 5 such as VF₃, VF₅, NbF₅, TaF₅, and so on; fluorides of Element 6 such as CrF₃, MoF₆, WF₆, and so on; fluorides of Element 7 such as MnF₂, MnF₃, ReF₆, and so on; fluorides of Element 8 such as FeF₃, RuF₃, RuF₄, OsF₄, OsF₅, OsF₆, and so on; fluorides of Element 9 such as CoF₂, CoF₃, RhF₃, IrF₆, and so on; fluorides of Element 10 such as NiF₂, PdF₂, PtF₂, PtF₄, PtF₆, and so on; fluorides of Element 11 such as CuF₂, CuFCl, AgF, AgF₂, and so on; fluorides of Element 12 such as ZnF₂, ZnFCl, CdF₂, HgF₂, and so on.

Suitable examples of mixture or compounds between or among the fluorides of typical elements and/or transition elements include, but are not limited to, HBF₄ [a compound of hydrogen fluoride (HF) and BF₃], HPF₆, HAsF₆, HSbF₆, LiF/HF [a mixture or salt of lithium fluoride(LiF) and hydrogen fluoride(HF)], NaF/HF, KF/HF, CsF/HF, (CH₃)₄NF/HF, (C₂H₅)₄NF/HF, (C₄H₉)₄NF/HF, ZnF₂/HF, CuF₂/HF, SbF₃/HF, SbF₅/HF, SbF₅/SbF₃, SbF₅/SbF₃/HF, ZnF₂/SbF₅, ZnF₂/SbF₅/HF, KF/SbF₅, KF/SbF₅/HF, and so on.

Suitable examples of mixtures, salts, or complexes of the fluorides with organic molecules include, but are not limited to, BF₃ diethyl etherate [BF₃.O(C₂H₅)₂], BF₃ dimethyl etherate, BF₃ dibutyl etherate, BF₃ tetrahydrofuran complex, BF₃ acetonitrile complex (BF₃.NCCH₃), BF₃.methanol complex, BF₃.ethanol complex, BF₃.propanol complex, HBF₄ diethyl etherate, HF/pyridine (a mixture of hydrogen fluoride and pyridine), HF/methylpyridine, HF/dimethylpyridine, HF/trimethylpyridine, HF/trimethylamine, HF/triethylamine, HF/dimethyl ether, HF/diethyl ether, and so on. As HF/pyridine, a mixture of about 70 wt % hydrogen fluoride and about 30 wt % pyridine is preferable due to availability of materials.

Among these examples of fluoride sources mentioned above, hydrogen fluoride, fluorides of the Elements 13˜15, fluorides of transition elements, and mixtures or compounds thereof, and mixtures, salts, or complexes of these fluorides with organic molecules are preferable.

Among the fluorides of transition elements, the fluorides of Elements 11 (Cu, Ag, Au) and 12 (Zn, Cd, Hg) are preferably exemplified. ZnF₂ and CuF₂ are furthermore preferable from the viewpoint of practical operation, yields, and cost. Among the fluorides of the Elements 13˜15, BF₃, AlF₃, AlF₂Cl, SbF₃, SbF₅, SbF₄Cl, and SbF₃Cl₂ are preferably exemplified. Fluorides of Elements 13˜15 can preferably be used for the preparation of polyfluorinated arylsulfur pentafluorides. Among the organic molecules usable for the mixtures, salts, or complexes with the fluorides, pyridine, ethers such as dimethyl ether, diethyl ether, dipropyl ether, and diisopropyl ether, alkylamines such as trimethylamine and triethylamine, and nitriles such as acetonitrile and propionitrile are preferable. Among these, pyridine, diethyl ether, triethylamine, and acetonitrile are more preferable because of availability and cost.

R¹, R², R³, R⁴, and/or R⁵ of the products represented by the formula (I) may be different from R¹, R², R³, R⁴, and/or R⁵ of the materials represented by the formula (II). Thus, embodiments of this invention include transformation of the R¹, R², R³, R⁴, and/or R⁵ to different R¹, R², R³, R⁴, and/or R⁵ which may take place during the reaction of the present invention or under the reaction conditions as long as the —SF₄X is transformed to a —SF₅ group.

In some cases, since the reaction of an arylsulfur halotetrafluoride and an anhydrous fluoride source is slowed down by flowing an inactive gas such as nitrogen (see Example 27), it is not preferable that the vapor on the reaction mixture and/or the gas which may be generated from the reaction mixture be removed, for example by flowing an inactive gas on or through the reaction mixture or other methods. Similarly, since the reaction with a fluoride source under hydrous conditions may be expected to be slowed down by flowing an inactive gas such as nitrogen, it may be preferable for the reactions under hydrous conditions to allow the vapor on the reaction mixture and/or the gas which may be generated from the reaction mixture to remain. Therefore, aspects of the invention include the reaction being carried out in a closed or sealed reactor, by using an autoclave as a reactor, by maintaining the reactor at a constant pressure, or by equipping the reactor with a balloon filled with an inactive gas such as nitrogen, or in any other like manner. In this manner, embodiments of the invention facilitate the presence of the reaction vapor.

Process I can be carried out in the presence of one or more solvent(s). However, in some embodiments, unlike most organic reactions, the present invention typically does not require a solvent. This presents an unexpected advantage to performing embodiments of the invention (due to lower cost, no solvent separating requirements, etc). In some cases, the use of solvent is preferable for mild and efficient reactions. Solvents herein may be hydrous or anhydrous (assuming other aspects or the reaction are hydrous). Where a solvent is utilized, alkanes, halocarbons, ethers, nitriles, nitro compounds can be used. Example alkanes include normal, branched, cyclic isomers of pentane, hexane, heptane, octane, nonane, decane, dodecan, undecane, and other like compounds. Illustrative halocarbons include; dichloromethane, chloroform, carbon tetrachloride, dichloroethane, trichloroethane, terachloroethane, trichlorotrifluoroethane, chlorobenzene, dichlorobenzene, trichlorobenzene, hexafluorobenzene, benzotrifluoride, and bis(trifluoromethyl)benzene; normal, branched, cyclic isomers of perfluoropentane, perfluorohexane, perfluorocyclohexane, perfluoroheptane, perfluorooctane, perfluorononane, and perfluorodecane; perfluorodecalin; and other like compounds. Illustrative ethers include diethyl ether, dipropyl ether, di(isopropyl) ether, dibutyl ether, tert-butyl methyl ether, dioxane, glyme (1,2-dimethoxyethane), diglyme, triglyme, and other like compounds. Illustrative nitriles include acetonitrile, propionitrile, benzonitrile, and other like compounds. Illustrative nitro compounds include nitromethane, nitroethane, nitrobenzene, and other like compounds. Where the fluoride source used for the reaction is liquid, it can be used as both a reactant and a solvent. A typical example of this is hydrogen fluoride or a mixture of hydrogen fluoride and pyridine. Hydrogen fluoride and a mixture of hydrogen fluoride and pyridine may also be used as a solvent.

In another embodiment, control of the reaction uses an arysulfur pentafluoride (of formula (I)) as a solvent for Process I, which is the reaction product, since separation of the product from a solvent is not required. For this embodiment, the lower amount of arylsulfur pentafluoride is preferable because of cost and effectiveness considerations.

Embodiments of this invention include hydrous conditions. In one embodiment an arylsulfur halotetrafluoride is reacted with a fluoride source under hydrous reaction conditions. The hydrous conditions can be provided in many ways to the reaction, including: (1) using a hydrous or moist fluoride source, as exemplified above; (2) using a wet or aqueous solvent as exemplified above, (3) using a wet or hydrous arylsulfur halotetrafluoride; (4) using water, steam, or water vapor added to the fluoride source, the arylsulfur halotetrafluoride, solvent, and/or reaction mixture or to the reaction system; (5) introducing moist or humid air to the fluoride source, the arylsulfur halotetrafluoride, solvent, and/or reaction mixture or reaction system; (6) using moist or humid air in which the reaction is run; and/or (7) using other like non-anhydrous conditions which can also be utilized to provide adequate hydrous conditions.

As the amount of water molecules in the reaction mixture increases, the amount of byproducts such as an arylsulfonyl fluoride having a formula (VI) may increase. However, the amount of the byproducts formed in the reaction may depend on the nature and/or quantity of the fluoride source(s), other starting materials, and on the reaction conditions. Therefore, the amount of water molecules used for the reactions herein may vary. However, in order to obtain good yield of products, the total amount of water in the reaction mixture or reaction system can preferably be chosen to be 50 weight % or less to a fluoride source used. More preferably, 25 weight % or less is chosen. Furthermore, 10 weight % or less, and more preferably, 5 weight % or less, furthermore preferably 3 weight % or less to the fluoride source used can be used herein. As such, hydrous conditions correspond to the weight % figures as defined above, but include each of the weight percent ranges.

In order to optimize yield with regard to Process I, the reaction temperature is selected in the range of from about −100° C. to about +250° C. More typically, the reaction temperature is selected in the range of from about −80° C. to about +230° C. Most typically, the reaction temperature is selected in the range of from about −60° C. to about +200° C.

In order to obtain economically good yields of the products, the amount of a fluoride source which provides n number of reactive fluoride (employable for the reaction) per molecule can be selected in the range of from about 1/n to about 20/n mol against about 1 mol of arylsulfur halotetrafluoride (see formula II). More typically, the amount can be selected in the range of from about 1/n to about 10/n mol from the viewpoint of yield and cost, as less amounts of a fluoride source decrease the yield(s) and additional amounts of a fluoride source do not significantly improve the yield(s).

The reaction time of Process I also varies dependent on reaction temperature, substrate identity, reagent identity, solvent identity, and their amounts used. Therefore, one can modify reaction conditions to determine the amount of time necessary for completing the reaction of Process I, but can be from about 1 minute to several days, preferably, within a few days.

In addition, the reaction(s) as described above can be conducted in any manner that will yield the products described herein. For example, reactants and a solvent(s) (if necessary) can be mixed and heated to a temperature at which the reaction proceeds; an arylsulfur halotetrafluoride can be gradually added to a mixture of other reactants and a solvent(s) (if necessary), which is heated at the temperature at which the reaction proceeds; or a fluoride source can be gradually added to a mixture of other reactants and a solvent(s) (if necessary) at the temperature at which the reaction proceedes. Under each condition, economically good yields are obtained showing the surprisingly utility of embodiments of the present invention.

The present invention also includes a process (Scheme 5, Process II) for preparing an arylsulfur pentafluoride having a formula (I), which comprises reacting an arylsulfur halotetrafluoride having a formula (II) with a fluoride source under hydrous conditions in the presence of a halogen, the halogen selected from the group of chlorine, bromine, iodine, and interhaloens.

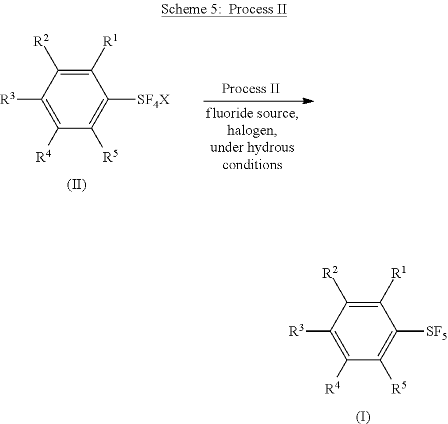

For formulas (I) and (II), R¹, R², R³, R⁴, R⁵, and X represent the same meaning as defined above, as does the definition of hydrous.

Process II is the same as Process I above except for the following modifications: the reaction of an arylsulfur halotetrafluoride and a fluoride source under hydrous conditions may be accelerated by including a halogen as selected from the group of chlorine, bromine, iodine, and interhalogens (see Example 26).

Typical halogens employable in Process II include chlorine (Cl₂), bromine (Br₂), iodine (I₂), and interhalogens such as ClF, BrF, ClBr, ClI, Cl₃I, and BrI. Among these, chlorine (Cl₂) is preferable due to its low cost.

R¹, R², R³, R⁴, and/or R⁵ of the products represented by the formula (I) may be different from R¹, R², R³, R⁴, and/or R⁵ of the materials represented by the formula (II). Thus, embodiments of this invention include transformation of the R¹, R², R³, R⁴, and/or R⁵ to different R¹, R², R³, R⁴, and/or R⁵ which may take place during the reaction of the present invention or under the reaction conditions as long as the —SF₄X is transformed to a —SF₅ group.

While not wanting to be tied to a particular mechanism, it is believed that the halogen activates a fluoride source and/or prevents reduction or disproportionation of an arylsulfur halotetrafluoride (formula II) which may occur during the reaction. Therefore, other fluoride source-activating and/or reduction or disproportionation-preventing compounds are within the scope of the invention. The reaction in the presence of the halogen may be carried out by methods such as by adding a halogen to the reaction mixture, dissolving a halogen in the reaction mixture, flowing a halogen gas or vapor into or onto the reaction mixture or the reactor, or others like means.

The amount of halogen used is from a catalytic amount to an amount in large excess. From the viewpoint of cost, a catalytic amount to about 5 mol of the halogen, can be preferably selected against about 1 mol of arylsulfur halotetrafluoride (formula II).

The present invention also includes processes (Scheme 6, Process I and Process III) for preparing an arylsulfur pentafluoride having a formula (I), which comprise reacting an arylsulfur halotetrafluoride having a formula (II) with a fluoride source under hydrous conditions and treating the resulting reaction products with hydrolysis conditions.

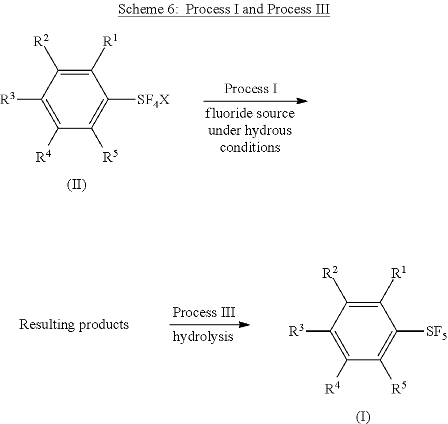

For formulas (I) and (II), R¹, R², R³, R⁴, R⁵, and X represent the same meaning as defined above, as well as the definition of hydrous.

Process I is described above. Process III is a known hydrolysis method; for example, hydrolysis in the presence or absence of a base or an acid with or without a phase-transfer catalyst. Preferably, hydrolysis in the presence of a base with or without a phase-transfer catalyst is conducted. Hydrolysis in the presence of a base with a phase-transfer catalyst is more preferable (See Example 21-24).

The products obtained by Process I or II may contain an arylsulfonyl fluoride having a formula (VI) as a byproduct;

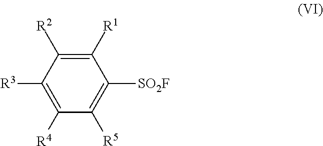

in some cases, it is not easy to remove the arylsulfonyl fluoride from the desired product, arylsulfur pentafluoride, because of no or a small difference in their properties such as solubility and boiling points. This invention includes a method of converting an arylsulfonyl fluoride of formula (VI) to an arylsulfonic acid or salt of formula (VII) in the presence of an arylsulfur pentafluoride of formula (I) as shown in the following reaction scheme 7:

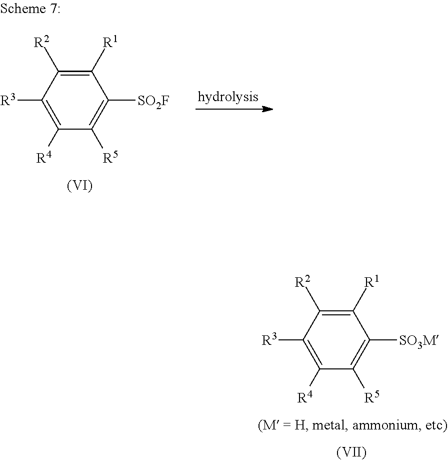

Since the properties (solubility, boiling points, melting points, etc.) of the arylsulfonic acid or salt are greatly different from those of the desired products, arylsulfur pentafluorides, separation of the arylsulfonic acid or salt from the arylsulfur pentafluoride is easy to obtain. For example, arylsulfonic acid or salt can easily be removed from the arylsulfur pentafluoride in a usual organic solvent by washing with water or alkaline.

In the processes I and III shown in Scheme 6, R¹, R², R³, R⁴, and/or R⁵ of the products represented by the formula (I) may be different from R¹, R², R³, R⁴, and/or R⁵ of the materials represented by the formula (II). Thus, embodiments of this invention include transformation of the R¹, R², R³, R⁴, and/or R⁵ to different R¹, R², R³, R⁴, and/or R⁵ which may take place during the reactions of the present invention or under the reaction conditions of Processes I and/or III as long as the —SF₄X is transformed to a —SF₅ group.

The present invention also includes a method (Scheme 8, Process II and Process III) for preparing an arylsulfur pentafluoride having a formula (I), which comprises reacting an arylsulfur halotetrafluoride having a formula (II) with a fluoride source under hydrous conditions in the presence of a halogen selected from the group of chlorine, bromine, iodine, and interhalogens, and treating the resulting reaction products with or under hydrolysis conditions.

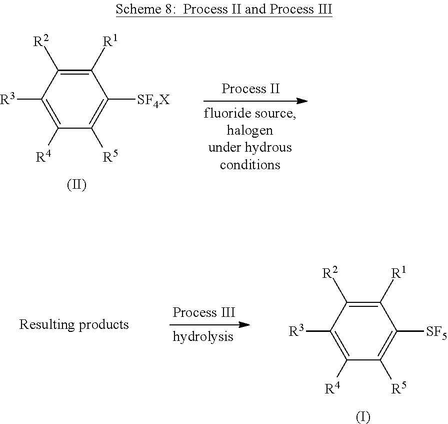

For formulas (I) and (II), R¹, R², R³, R⁴, R⁵, and X represent the same meaning as defined above, as is the definition for hydrous.

Processes II and III are described above.

In the processes shown in Scheme 8, R¹, R², R³, R⁴, an/or R⁵ of the products represented by the formula (I) may be different from R¹, R², R³, R⁴, and/or R⁵ of the materials represented by the formula (II). Thus, embodiments of this invention include transformation of the R¹, R², R³, R⁴, and/or R⁵ to different R¹, R², R³, R⁴, and/or R⁵ which may take place during the reactions of the present invention or under the reaction conditions of Processes II and/or III as long as the —SF₄X is transformed to a —SF₅ group.

The present invention provides a purification method for an arylsulfur pentafluoride of formula (I) by treating a mixture having the arylsulfur pentafluoride under hydrolysis conditions. This hydrolysis can be conducted through any number of known hydrolysis conditions, for example, hydrolysis in the presence or absence of a base or an acid with or without a phase-transfer catalyst. Preferably, hydrolysis in the presence of a base with or without a phase-transfer catalyst is conducted. Hydrolysis in the presence of a base with a phase-transfer catalyst is more preferable. This method is to remove an arylsulfonyl fluoride of formula (VI) from the arylsulfur pentafluoride that coexists with the arylsulfonyl fluoride. The arylsulfonyl fluorides are hydrolyzed to arylsulfonic acids or salts which are easily separated from the arylsulfur pentafluorides.

According to the present invention, the arylsulfur pentafluorides having the formula (I) can be easily and cost-effectively produced from available starting materials and reagents.

The following examples will illustrate the present invention in more detail, but it should be understood that the present invention is not deemed to be limited thereto.

### EXAMPLES

The following examples are provided for illustrative purposes only and are not intended to limit the scope of the invention. Tables 2 and 3 provide chemical names and their chemical structures and formula numbers for reference, which can be synthesized as products or can be used as starting materials in the present invention.

TABLE 2
Arylsulfur pentafluorides (Formula I):

| Formula | | |
|---|---|---|
| Number | Name | Structure |
| Ia | Phenylsulfur pentafluoride | 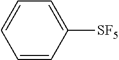 |
| Ib | p-Methylphenylsulfur pentafluoride | 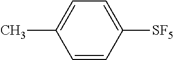 |
| Id | p-Fluorophenylsulfur pentafluoride | 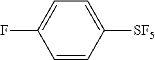 |
| Ie | o-Fluorophenylsulfur pentafluoride | 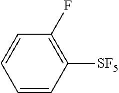 |
| If | p-Chlorophenylsulfur pentafluoride | 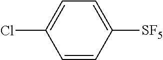 |
| Ig | p-Bromophenylsulfur pentafluoride | 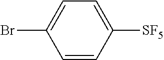 |
| Ih | m-Bromophenylsulfur pentafluoride | 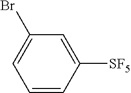 |
| Ii | p-Nitrophenylsulfur pentafluoride | 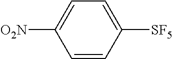 |
| Ij | 2,6- Difluorophenylsulfur pentafluoride | 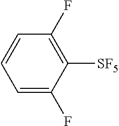 |
| Ik | 3-Chloro-2,6- difluorophenylsulfur pentafluoride | 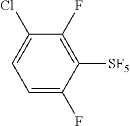 |
| Il | 2,4,6- Trifluorophenylsulfur pentafluoride | 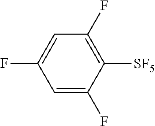 |
| Im | 3-Chloro-2,4,6- trifluorophenylsulfur pentafluoride | 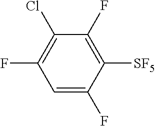 |
| In | 2,3,4,5,6- Pentafluorophenylsulfur pentafluoride | 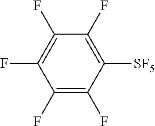 |

TABLE 3
Arylsulfur halotetrafluorides (Formula II)

| Formula | | |
|---|---|---|
| Number | Name | Structure |
| IIa | Phenylsulfur chlorotetrafluoride | 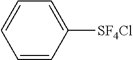 |
| IIb | p-Methylphenylsulfur chlorotetrafluoride | 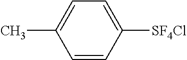 |
| IIc | p-(tert-Butyl)phenylsulfur chlorotetrafluoride | 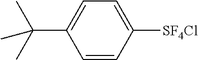 |
| IId | p-Fluorophenylsulfur chlorotetrafluoride | 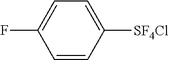 |
| IIe | o-Fluorophenylsulfur chlorotetrafluoride | 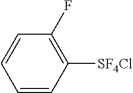 |
| IIf | p-Chlorophenylsulfur chlorotetrafluoride | 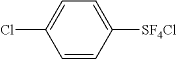 |
| IIg | p-Bromophenylsulfur chlorotetrafluoride | 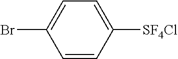 |
| IIh | m-Bromophenylsulfur chlorotetrafluoride | 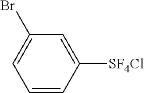 |
| IIi | p-Nitrophenylsulfur chlorotetrafluoride | 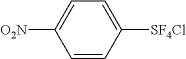 |
| IIj | 2,6- Difluorophenylsulfur chlorotetrafluoride | 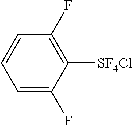 |
| IIl | 2,4,6- Trifluorophenylsulfur chlorotetrafluoride | 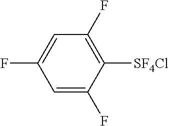 |
| lln | 2,3,4,5,6- Pentafluorophenylsulfur chlorotetrafluoride | 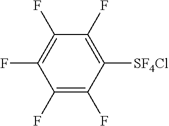 |

### Example 1

### Preparation of phenylsulfur chlorotetrafluoride (IIa)

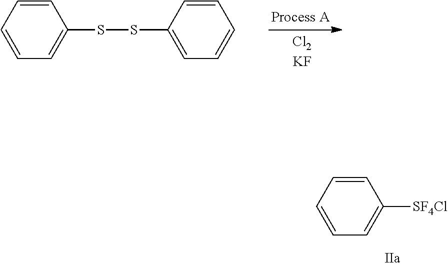

(Process A) A 500 mL round bottom glassware flask was charged with diphenyl disulfide (33.0 g, 0.15 mol), dry potassium fluoride (KF) (140 g, 2.4 mol) and 300 mL of dry acetonitrile. The stirred reaction mixture was cooled on an ice/water bath under a flow of nitrogen (N₂) (18 mL/min). After N₂ was stopped, chlorine (Cl₂) was bubbled into a reaction mixture at the rate of about 70 mL/min. The Cl₂ bubbling took about 6.5 h. The total amount of Cl₂ used was about 1.2 mol. After Cl₂ was stopped, the reaction mixture was stirred for additional 3 h. N₂ was then bubbled through for 2 hours to remove an excess of Cl₂. The reaction mixture was then filtered with 100 mL of dry hexanes in air. About 1 g of dry KF was added to the filtrate. The KF restrains possible decomposition of the product. The filtrate was evaporated under vacuum and the resulting residue was distilled at reduced pressure to give a colorless liquid (58.0 g, 88%) of phenylsulfur chlorotetrafluoride (IIa): b.p. 80° C./20 mmHg; ¹H NMR (CD₃CN) δ 7.79-7.75 (m, 2H, aromatic), 7.53-7.49 (m, 3H, aromatic); ¹⁹F NMR (CD₃CN) δ 136.7 (s, SF₄Cl). The NMR analysis showed phenylsulfur chlorotetrafluoride obtained is a trans isomer.

### Examples 2-16

### Preparation of arylsulfur chlorotetrafluorides (IIa˜j,l,n) from the Corresponding aryl sulfur Compounds (IIIa) or (IIIb) or arylsulfur trifluorides (V)

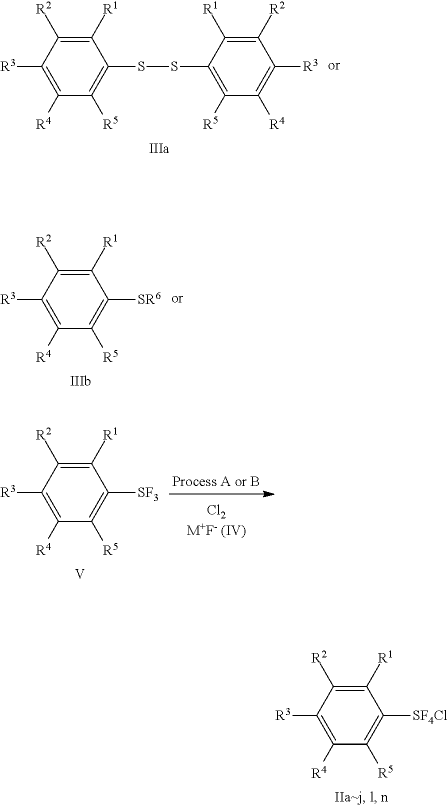

Substituted and unsubstituted arylsulfur chlorotetrafluorides (IIa˜j,l,n) were synthesized from the corresponding aryl sulfur compounds (IIIa) or (IIIb) or arylsulfur trifluorides (V) according to a similar procedure as described in Example 1. Table 4 shows the synthesis of the substituted and unsubstituted arylsulfur chlorotetrafluorides. Table 4 also shows the starting materials and other chemicals, solvents, reaction conditions, and results, together with those of Example 1.

TABLE 4
Preparation of arylsulfur chlorotetrafluorides (IIa~j,l,n) from aryl sulfur
compounds (IIIa) or (IIIb) or arylsulfur trifluorides (V)

| Ex. | (IIa) or (IIIb) or (V) | Halogen | (IV) | Solvent | Conditions | (II) | Yield |
|---|---|---|---|---|---|---|---|
| 1 | 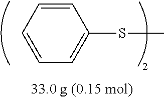 | Cl₂ ~1.2 mol | KF 140 g (2.4 mol) | CH₃CN 300 mL | 0~5° C. ~9.5 h | 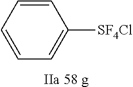 | 88% |
| 2 | 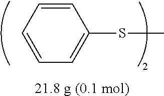 | Cl₂ 0.68 mol | CsF 243 g (1.6 mol) | CH₃CN 200 mL | 0~5° C. 4 h and r.t. overnight | 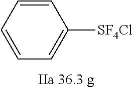 | 83% |
| 3 | 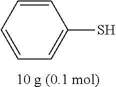 | Cl₂ 0.68 mol | KF 47.5 g (0.817 mol) | CH₃CN 100 mL | 6~10° C. 3.7 h | 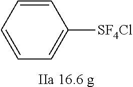 | 83% |
| 4 | 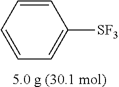 | Cl₂ 65.5 mmol | CsF 8.74 g (150 mmol) | CH₃CN 20 mL | 6~9° C. 0.7 h | 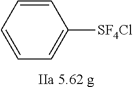 | 84% |
| 5 | 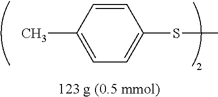 | Cl₂ 0.73 mol | KF 464 g (8 mol) | CH₃CN 1 L | 0° C. 10.5 h | 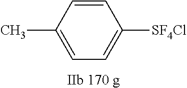 | 73% |
| 6 | 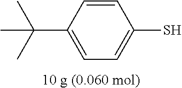 | Cl₂ 0.45 mol | CsF 91.6 g (0.602 mol) | CH₃CN 150 mL | 5~10° C. 3.5 h and r.t. 24 h | 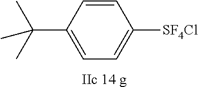 | 84% |
| 7 | 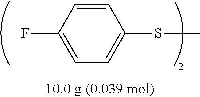 | Cl₂ 0.28 mol | KF 36 g (0.63 mol) | CH₃CN 100 mL | 0~5° C. 2.5 h and r.t. overnight | 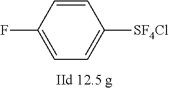 | 67% |
| 8 | 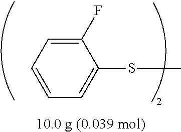 | Cl₂ 0.31 mol | KF 36.5 g (0.63 mol) | CH₃CN 100 mL | 0~5° C. 1.8 h and r.t. overnight | 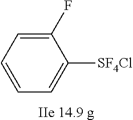 | 80% |
| 9 | 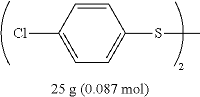 | Cl₂ 0.57 mol | KF 86 g (1.48 mol) | CH₃CN 200 mL | 5~8° C. 3.5 h | 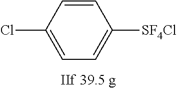 | 88% |
| 10 | 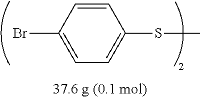 | Cl₂ 0.72 mol | KF 94 g (1.6 mol) | CH₃CN 200 mL | 0~5° C. 4.5 h and r.t. overnight | 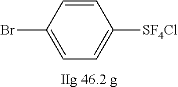 | 77% |
| 11 | 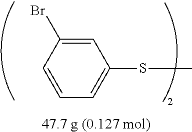 | Cl₂ 0.88 mol | KF 118 g (2.0 mol) | CH₃CN 250 mL | 0~5° C. 5.5 h and r.t. overnight | 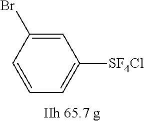 | 86% |
| 12 | 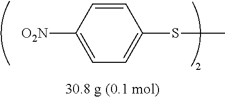 | Cl₂ 0.72 mol | KF 94 g (1.6 mol) | CH₃CN 200 mL | 0~5° C. 4.5 h and r.t. overnight | 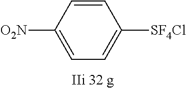 | 60% |
| 13 | 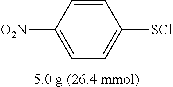 | Cl₂ 0.72 mol | KF 15.3 g (264 mmol) | CH₃CN 40 mL | 5~11° C. 1.1 h | 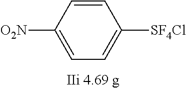 | 76% |
| 14 | 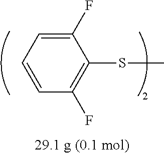 | Cl₂ ~1.02 mol | CsF 279 g (1.83 mol) | CH₃CN 200 mL | 0~5° C. 5 h and r.t. overnight | 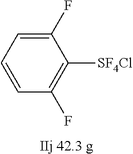 | 82% |
| 15 | 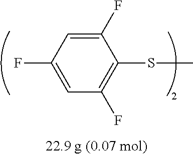 | Cl₂ ~1.08 mol | KF 90 g (1.55 mol) | CH₃CN 300 mL | 0~5° C. 6 h and r.t. overnight | 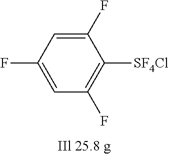 | 67% |
| 16 | 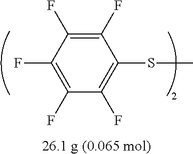 | Cl₂ ~1 mol | KF 82 g (1.41 mol) | CH₃CN 300 mL | 0~5° C. 5 h and r.t. overnight | 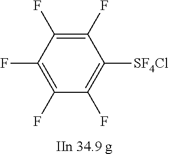 | 86% |

The properties and spectral data of the product (IIa) obtained by Examples 2-4 are shown in Example 1. The properties and spectral data of the products obtained by Examples 5-16 are shown by the following:

p-Methylphenylsulfur chlorotetrafluoride (IIb); b.p. 74-75° C./5 mmHg; ¹H NMR (CD₃CN) δ 7.65 (d, 2H, aromatic), 7.29 (d, 2H, aromatic), 2.36 (s, 3H, CH₃); ¹⁹F NMR (CD₃CN) δ 137.66 (s, SF₄Cl); High resolution mass spectrum; found 235.986234 (34.9%) (calcd for C₇H₇F₄S³⁷Cl; 235.986363), found 233.989763 (75.6%) (calcd for C₇H₇F₄S³⁵Cl; 233.989313). The NMR shows that p-methylphenylsulfur chlorotetrafluoride obtained is a trans isomer.

p-(tert-Butyl)phenylsulfur chlorotetrafluoride (IIc); b.p. 98° C./0.3 mmHg; m.p. 93° C.; ¹H NMR (CDCl₃) δ 1.32 (s, 9H, C(CH₃)₃), 7.43 (d, J=9.2 Hz, 2H, aromatic), 7.64 (d, J=9.2 Hz, 2H, aromatic); ¹⁹F NMR δ 138.3 (s, SF₄Cl). High resolution mass spectrum; found 278.034576 (8.8%) (calcd for C₁₀H₁₃³⁷ClF₄S; 278.033313), found 276.037526 (24.7%) (calcd for C₁₀H₁₃³⁵ClF₄S; 276.036263). Elemental analysis; Calcd for C₁₀H₁₃ClF₄S; C, 43.40%; H, 4.74%. Found; C, 43.69%, H, 4.74%. The NMR showed that p-(tert-butyl)phenylsulfur chlorotetrafluoride was obtained as a trans isomer.

p-Fluorophenylsulfur chlorotetrafluoride (IId); b.p. 60° C./8 mmHg; ¹H NMR (CD₃CN) δ 7.85-7.78 (m, 2H, aromatic), 7.25-7.15 (m, 2H, aromatic); ¹⁹F NMR (CD₃CN) δ 137.6 (s, SF₄Cl), −108.3 (s, CF); High resolution mass spectrum; found 239.961355 (37.4%) (calcd for C₆H₄F₅S³⁷Cl; 239.961291), found 237.964201(100%) (calcd for C₆H₄F₅S³⁵Cl; 237.964241). The NMR shows that p-fluorophenylsulfur chlorotetrafluoride obtained is a trans isomer.

o-Fluorophenylsulfur chlorotetrafluoride (IIe); b.p. 96-97° C./20 mmHg; ¹H NMR (CD₃CN) δ 7.77-7.72 (m, 1H, aromatic), 7.60-7.40 (m, 1H, aromatic), 7.25-7.10 (m, 2H, aromatic); ¹⁹F NMR (CD₃CN) δ 140.9 (d, SF₄Cl), −107.6 (s, CF); High resolution mass spectrum; found 239.961474 (25.4%) (calcd for C₆H₄F₅S³⁷Cl; 239.961291), found 237.964375 (69.8%) (calcd for C₆H₄F₅S³⁵Cl; 237.964241). The NMR shows that o-fluorophenylsulfur chlorotetrafluoride obtained is a trans isomer.

p-Chlorophenylsulfur chlorotetrafluoride (IIf); b.p. 65-66° C./2 mmHg; ¹H NMR (CDCl₃) δ 7.38 (d, 2H, J=9.1 Hz), 7.65 (d, 2H, J=9.1 Hz); ¹⁹F NMR (CDCl₃) δ 137.4 (s, 4F, SF₄Cl). High resolution mass spectrum; found 257.927507 (13.3%) (calcd for C₆H₄F₄S³⁷Cl₂; 257.928790), found 255.930746 (68.9%) (calcd for C₆H₄F₄S³⁷Cl³⁵Cl; 255.931740), found 253.933767 (100.0%) (calcd for C₆H₄F₄S³⁵Cl₂; 253.934690). The NMR showed that p-chlorophenylsulfur chlorotetrafluoride obtained is a trans isomer.

p-Bromophenylsulfur chlorotetrafluoride (IIg); m.p. 58-59° C.; ¹H NMR (CD₃CN) δ 7.67 (s, 4H, aromatic); ¹⁹F NMR (CD₃CN) δ 136.56 (s, SF₄Cl); High resolution mass spectrum; found 301.877066 (16.5%) (calcd for C₆H₄⁸¹Br³⁷ClF₄S; 301.879178), found 299.880655 (76.6%) (calcd for C₆H₄⁸¹Br³⁵ClF₄S; 299.881224 and calcd for C₆H₄⁷⁹Br³⁷ClF₄S; 299.882128), found 297.882761 (77.4%) (calcd for C₆H₄⁷⁹Br³⁵ClF₄S; 297.884174). Elemental analysis; calcd for C₆H₄BrClF₄S; C, 24.06%; H, 1.35%; found, C, 24.37%; H, 1.54%. The NMR showed that p-bromophenylsulfur chlorotetrafluoride was obtained as a trans isomer.

m-Bromophenylsulfur chlorotetrafluoride (IIh); b.p. 57-59° C./0.8 mmHg; ¹H NMR (CD₃CN) δ 7.90-7.88 (m, 1H, aromatic), 7.70-7.50 (m, 2H, aromatic), 7.40-7.30 (m, 1H, aromatic); ¹⁹F NMR (CD₃CN) δ 136.74 (s, SF₄Cl). High resolution mass spectrum; found 301.878031 (29.1%) (calcd for C₆H₄⁸¹Br³⁷ClF₄S; 301.879178), found 299.881066 (100%) (calcd for C₆H₄⁸¹Br³⁵ClF₄S; 299.881224 and calcd for C₆H₄⁷⁹Br³⁷ClF₄S; 299.882128), found 297.883275 (77.4%) (calcd for C₆H₄⁷⁹Br³⁵ClF₄S; 297.884174). The NMR showed that m-bromophenylsulfur chlorotetrafluoride obtained was a trans isomer.

p-Nitrophenylsulfur chlorotetrafluoride (IIi); m.p. 130-131° C.; ¹H NMR (CD₃CN) δ 8.29 (d, J=7.8 Hz, 2H, aromatic), 8.02 (d, J=7.8 Hz, 2H, aromatic); ¹⁹F NMR (CD₃CN) δ 134.96 (s, SF₄Cl); High resolution mass spectrum; found 266.956490 (38.4%) (calcd for C₆H₄³⁷ClF₄NO₂S; 266.955791), found 264.959223 (100%) (calcd for C₆H₄³⁵ClF₄NO₂S; 264.958741). Elemental analysis; calcd for C₆H₄ClF₄NO₂S; C, 27.13%; H, 1.52%; N, 5.27%; found, C, 27.16%; H, 1.74%; N, 4.91%. The NMR shows that p-nitrophenylsulfur chlorotetrafluoride obtained is a trans isomer.

2,6-Difluorophenylsulfur chlorotetrafluoride (IIj): The product (b.p. 120-122° C./95-100 mmHg) obtained from Example 14 is a 6:1 mixture of trans- and cis-isomers of 2,6-difluorophenylsulfur chlorotetrafluoride. The trans-isomer was isolated as pure form by crystallization; mp. 47.6-48.3° C.; ¹⁹F NMR (CDCl₃) δ 143.9 (t, J=26.0 Hz, 4F, SF₄), −104.1 (quintet, J=26.0 Hz, 2F, 2.6-F): ¹H NMR (CDCl₃) δ 6.97-7.09 (m, 2H, 3.5-H), 7.43-7.55 (m, 1H, 4-H); ¹³C NMR (CDCl₃) δ 157.20 (d, J=262.3 Hz), 133.74 (t, J=11.6 Hz), 130.60 (m), 113.46 (d, J=14.6 Hz); high resolution mass spectrum; found 257.950876 (37.6%) (calcd for C₆H₃³⁷ClF₆S; 257.951869), found 255.955740 (100%) (calcd for C₆H₃³⁵ClF₆S; 255.954819); elemental analysis; calcd for C₆H₃ClF₆S; C, 28.08%, H, 1.18%; found; C, 28.24%, H, 1.24%. The cis-isomer was assigned in the following; ¹⁹F NMR (CDCl₃) δ 158.2 (quartet, J=161.8 Hz, 1F, SF), 121.9 (m, 2F, SF₂), 76.0 (m, 1F, SF). The ¹⁹F NMR assignment of aromatic fluorine atoms of the cis-isomer could not be done because of possible overlapping of the peaks of the trans-isomer.

2,4,6-Trifluorophenylsulfur chlorotetrafluoride (IIl): trans-isomer; m.p. 55.8-56.7° C.; ¹⁹F NMR (CDCl₃) δ 144.07 (t, J=26.0 Hz, 4F, SF₄), −99.80 (t, J=26.0 Hz, 2F, o-F), −100.35 (s, 1F, p-F); ¹H NMR (CDCl₃) δ 6.79 (t, J=17.5 Hz, m-H); ¹³C NMR (CDCl₃) δ 164.16 (dt, J=164.2 Hz, 15.2 Hz, 4-C), 158.18 (dm, J=260.7 Hz, 2-C), 127.7 (m, 1-C), 102.1 (tm, J=27.8 Hz, 3-C). Elemental analysis; calcd for C₆H₂ClF₇S; C, 26.24%; H, 0.73%; found C, 26.23%; H, 1.01%. The NMR shows that 2,4,6-trifluorophenylsulfur chlorotetrafluoride obtained is a trans isomer.

2,3,4,5,6-Pentafluorophenylsulfur chlorotetrafluoride (IIn): The product (b.p. 95-112° C./100 mmHg) obtained from Experiment 16 was a 1.7:1 mixture of trans and cis isomers of 2,3,4,5,6-pentafluorophenylsulfur chlorotetrafluoride. The isomers were assigned by ¹⁹F NMR: The trans isomer; ¹⁹F NMR (CDCl₃) δ 144.10 (t, J=26.0 Hz, 4F, SF₄), −132.7 (m, 2F, 2.6-F), −146.6 (m, 1F, 4-F), −158.9 (m, 2F, 3.5-F); ¹³C NMR (CDCl₃) δ 143.5 (dm, J=265.2 Hz), 141.7 (dm, J=263.7 Hz), 128.3 (m). The cis isomer; ¹⁹F NMR (CDCl₃) δ 152.39 (quartet, J=158.9 Hz, 1F, SF), 124.32 (m, 2F, SF₂), 79.4 (m, 1F, SF), −132.7 (m, 2F, 2.6-F), −146.6 (m, 1F, 4-F), −158.9 (m, 2F, 3.5-F). High resolution mass spectrum of a 1.7:1 mixture of the trans and cis isomers; found 311.923124 (15.5%) (calcd for C₆³⁷ClF₉S; 311.923604), found 309.926404 (43.1%) (calcd for C₆³⁵ClF₉S; 309.926554).

### Example 17

### Reaction of phenylsulfur chlorotetrafluoride with SbF₃ Containing 1.5 wt % Water at Room Temperature

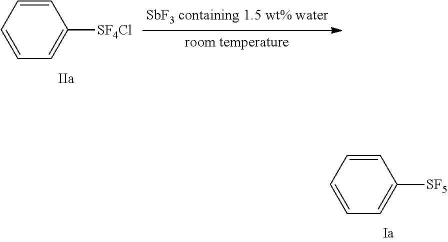

A reaction vessel made of fluoropolymer (PFA) was charged with 4.94 g (SbF₃; 27.2 mmol) of SbF₃ containing 1.5 wt % water and 5.0 g (22.7 mmol) of trans-phenylsulfur chlorotetrafluoride (trans-PhSF₄Cl). The SbF₃ containing 1.5 wt % water was prepared by adding the amount of water necessary for the content to anhydrous SbF₃ just before the reaction. The vessel was equipped with a balloon filled with N₂. The reaction mixture was stirred at room temperature and monitored by ¹⁹F-NMR. Some of trans-PhSF₄Cl isomerized to cis-PhSF₄Cl during reaction. The molar ratio of the product (PhSF₅): PhSF₄Cl (a mixture of trans- and cis-isomers) was determined by ¹⁹F NMR at 2 hours, 3.5 hours, and 18.5 hours reaction time. The reaction was completed within 18.5 hours and phenylsulfur pentafluoride (PhSF₅) was produced in 49% yield and phenylsulfonyl fluoride (PhSO₂F) was formed in 13% yield as a byproduct. The results are shown in Table 5 below.

TABLE 5
Reaction of phenylsulfur chlorotetrafluoride with
SbF₃ containing 1.5 wt % water at room temperature

| Reaction | Molar ratio of | Yield of | Yield of |
|---|---|---|---|
| time | PhSF₅:PhSF₄Cl | PhSF₅ | PhSO₂F |
| 2 hours | 1:1.3 | — | — |
| 3.5 hours | 1:0.07 | — | — |
| 18.5 hours | No PhSF₄Cl | 49% | 13% |

Properties and spectral data of phenylsulfur pentafluoride obtained, are as follows: b.p. 70-71° C./120 mmHg; ¹H NMR(CDCl₃) δ 7.77-7.74 (m, 2H, aromatic), 7.60-7.40 (m, 3H, aromatic); ¹⁹F NMR (CDCl₃) δ 85.20-84.13 (m, 1F, SF₅), 62.91 (d, 4F, SF₅).

### Comparative Example 1

### Reaction of phenylsulfur chlorotetrafluoride with anhydrous SbF₃ at Room Temperature

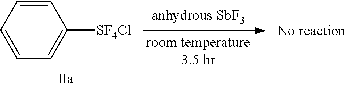

A reaction vessel made of fluoropolymer (PFA) was charged with 4.90 g (27.4 mmol) of anhydrous SbF₃ and 5.01 g (22.7 mmol) of trans-phenylsulfur chlorotetrafluoride. The reaction vessel was equipped with a balloon filled with N₂. The reaction mixture was stirred at room temperature and monitored by ¹⁹F-NMR at 2 hours and 3.5 hours reaction time. The results are shown in Table 6 below.

TABLE 6
Reaction of phenylsulfur chlorotetrafluoride
with anhydrous SbF₃ at room temperature

| Reaction | |
|---|---|
| time | |
| 2 hours | No reaction |
| 3.5 hours | No reaction |

As shown in Table 6, the reaction of phenylsulfur chlorotetrafluoride with anhydrous SbF₃ at room temperature did not occur even after 3.5 hours. On the other hand, the reaction with SbF₃ under hydrous conditions occurred smoothly at room temperature and almost to completion in 3.5 hours as seen in Table 5 (Example 17). Thus, the hydrous reaction conditions remarkably accelerate the fluorination reaction compared to the anhydrous reaction conditions.

### Example 18

### Reaction of phenylsulfur chlorotetrafluoride with ZnF₂ Containing 1.5 wt % Water at 100° C.

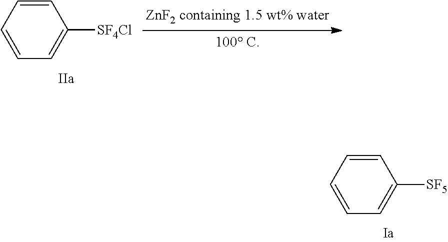

A reaction vessel made of fluoropolymer was charged with 1.55 g (ZnF₂; 67.3 mmol) of ZnF₂ containing 1.5 wt % water and 4.96 g (22.5 mmol) of trans-phenylsulfur chlorotetrafluoride (trans-PhSF₄Cl). The ZnF₂ containing 1.5 wt % water was prepared by adding the amount of water necessary for the content to anhydrous ZnF₂. The vessel was equipped with a condenser made of fluoropolymer and a balloon filled with N₂. The reaction mixture was stirred at 100° C. and monitored by ¹⁹F-NMR. Some of trans-PhSF₄Cl isomerized to cis-PhSF₄Cl during reaction. The molar ratio of the product (PhSF₅): PhSF₄Cl (a mixture of trans- and cis-isomers) was determined by ¹⁹F NMR at 2 hours and 5 hours reaction time. The results are shown in Table 7 below.

TABLE 7
Reaction of phenylsulfur chlorotetrafluoride
with ZnF₂ containing 1.5 wt % water at 100° C.

| Reaction | Molar ratio of |
|---|---|
| time | PhSF₅:PhSF₄Cl |
| 2 hours | 1:3.7 |
| 5 hours | 1:0.9 |

### Comparative Example 2

### Reaction of phenylsulfur chlorotetrafluoride with anhydrous ZnF₂ at 100° C.

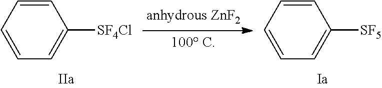

A reaction vessel made of fluoropolymer was charged with 1.51 g (14.7 mmol) of anhydrous ZnF₂ and 5.0 g (22.7 mmol) of trans-phenylsulfur chlorotetrafluoride (trans-PhSF₄Cl). The vessel was equipped with a condenser made of fluoropolymer and a balloon filled with N₂. The reaction mixture was stirred at 100° C. and monitored by ¹⁹F NMR. Some of trans-PhSF₄Cl isomerized to cis-PhSF₄Cl during reaction. The molar ratio of the product (PhSF₅): PhSF₄Cl (a mixture of trans- and cis-isomers) was determined by ¹⁹F NMR at 2 hours and 5 hours reaction time. The results are shown in Table 8 below.

TABLE 8
Reaction of phenylsulfur chlorotetrafluoride
with anhydrous ZnF₂ at 100° C.

| Reaction | Molar ratio of |
|---|---|
| time | PhSF₅:PhSF₄Cl |
| 2 hours | 1:15 |
| 5 hours | 1:9 |

The comparison of Example 18 and Comparative Example 2 showed that hydrous reaction conditions can remarkably accelerate the fluorination reaction compared to anhydrous conditions.

### Example 19

### Synthesis of phenylsulfur pentafluoride from phenylsulfur chlorotetrafluoride with ZnF₂ Containing 1.5wt % Water at 120° C.

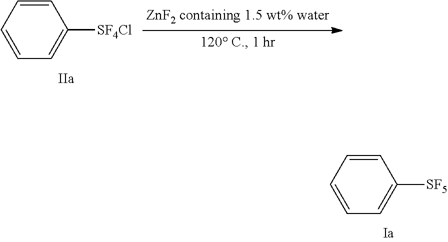

A reaction vessel made of fluoropolymer was charged with 1.54 g (ZnF₂; 14.8 mmol) of ZnF₂ containing 1.5 wt % water and 4.99 g (22.6 mmol) of trans-phenylsulfur chlorotetrafluoride. The ZnF₂ containing 1.5 wt % water was prepared by adding the amount of water necessary for the content to anhydrous ZnF₂. The vessel was equipped with a condenser made of fluoropolymer and a balloon filled with N₂. The reaction mixture was stirred at 120° C. for 1 hour. The reaction was completed in 1 hour. ¹⁹F-NMR analysis of the reaction mixture showed that phenylsulfur pentafluoride was produced in 89% yield. Phenylsulfonyl fluoride as a byproduct was produced in 2% yield.

### Example 20

### Synthesis of phenylsulfur pentafluoride from phenylsulfur chlorotetrafluoride with ZnF₂ Containing 4 wt % ZnF₂(H₂O)ₙ (n=about 3˜4)

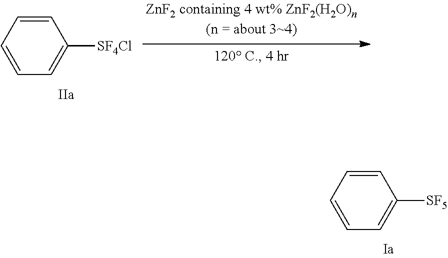

A reaction vessel made of fluoropolymer was charged with 1.56 g (ZnF₂; 14.9 mmol) of ZnF₂ containing 4 wt % ZnF₂(H₂O)ₙ (n=about 3˜4) and 5.0 g (22.7 mmol) of trans-phenylsulfur chlorotetrafluoride. The ZnF₂ containing 4 wt % ZnF₂(H₂O)ₙ (n=about 3˜4) was prepared by adding the amount of ZnF₂ hydrate [ZnF₂(H₂O)ₙ (n=about 3-4)] (from Sigma-Aldrich) necessary for the content to anhydrous ZnF₂. The vessel was equipped with a condenser made of fluoropolymer and a balloon filled with N₂. The reaction mixture was stirred at 120° C. for 4 hour. ¹⁹F-NMR analysis of the reaction mixture showed that phenylsulfur pentafluoride was produced in 83% yield. Phenylsulfonyl fluoride was detected in a trace amount by the NMR.

### Example 21

### Synthesis of p-methylphenylsulfur pentafluoride from p-methylphenylsulfur chlorotetrafluoride with hydrous hydrogen fluoride-pyridine

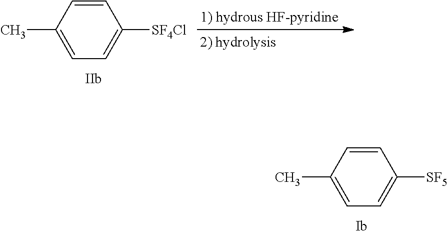

A reaction vessel made of fluoropolymer was charged with 1.0 g (4.26 mmol) of trans-p-methylphenylsulfur chlorotetrafluoride (trans-p-MeC₆H₄SF₄Cl), and 0.55 mL of a mixture of 98.5 wt % hydrogen fluoride-pyridine (HF:pyridine=about 7:3 weight ratio) and 1.5 wt % water was added at room temperature. A mixture of 98.5 wt % hydrogen fluoride-pyridine (HF:pyridine=about 7:3 weight ratio) and 1.5 wt % water was prepared by adding the amount of water necessary for the content to a mixture of anhydrous hydrogen fluoride-pyridine (HF:pyridine=about 7:3 weight ratio) (from Sigma-Aldrich). The vessel was equipped with a balloon filled with N₂. The reaction mixture was stirred at room temperature for 24 hours. At this time, the reaction was completed (p-methylphenylsulfur chlorotetrafluoride was consumed). An analysis of the reaction mixture by ¹⁹F-NMR showed that p-methylphenylsulfur pentafluoride (p-MeC₆H₄SF₅) was produced in 65% yield and p-methylphenylsulfonyl fluoride (p-MeC₆H₄SO₂F) was produced in 5% yield as a byproduct.

TABLE 9
Reaction of p-methylphenylsulfur chlorotetrafluoride
with hydrous HF-pyridine at room temperature

| | Molar ratio of | | |
|---|---|---|---|
| Reaction | p-MeC₆H₄SF₅: | Yield of | Yield of |
| time | p-MeC₆H₄SF₄Cl | p-MeC₆H₄SF₅ | p-MeC₆H₄SO₂F |
| 24 hours | p-MeC₆H₄SF₄Cl | 65% | 5% |
| | was consumed | | |

The reaction mixture was neutralized and extracted with diethyl ether. The ether solution (15 mL) was treated with 8.5 mL of 2.5 N aq. sodium hydroxide solution which contained benzyltrimethylammonium chloride (5 mol % to NaOH) (as a phase-transfer catalyst) at room temperature for 9 hours, leaving p-methylphenylsulfur pentafluoride as an only product quantitatively. All the p-methylphenylsulfonyl fluoride was hydrolyzed to p-methylphenylsulfonic acid salt and removed to an aqueous layer.

Properties and spectral data of p-methylphenylsulfur pentafluoride obtained are as follows; b.p. 95-96° C./80 mmHg; ¹H NMR (CDCl₃) δ 7.63 (d, 2H, aromatic), 7.24 (d, 2H, aromatic), 2.40 (s, 3H, CH₃); ¹⁹F NMR (CDCl₃) δ 86.55-84.96 (m, 1F, SF), 63.26 (d, 4F, SF₄).

### Comparative Example 3

### Reaction of p-methylphenylsulfur chlorotetrafluoride with anhydrous hydrogen fluoride-pyridine

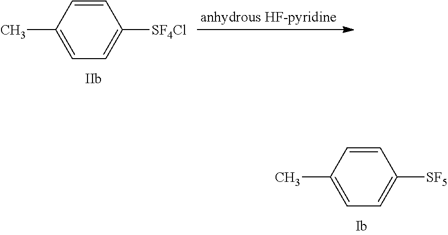

The reaction was carried out at room temperature in the same manner as in the first reaction of Example 21 except that 0.55 mL of anhydrous hydrogen fluoride-pyridine (HF:pyridine=about 7:3 weight ratio) (from Sigma-Aldrich) was used in place of 0.55 mL of a mixture of 98.5 wt % hydrogen fluoride-pyridine (HF:pyridine=about 7:3 weight ratio) and 1.5 wt % water. The reaction was monitored with ¹⁹F-NMR. Some of trans-p-MeC₆H₄SF₄Cl isomerized to cis-p-MeC₆H₄SF₄Cl during reaction. The molar ratio of the product (p-MeC₆H₄SF₅): p-MeC₆H₄SF₄Cl (a mixture of trans- and cis-isomers) was determined at different reaction times and the results are shown in Table 10 below.

TABLE 10
Reaction of p-methylphenylsulfur chlorotetrafluoride
with anhydrous HF-pyridine at room temperature

| | Molar ratio of | |
|---|---|---|
| Reaction | p-MeC₆H₄SF₅: | Yield of |
| time | p-MeC₆H₄SF₄Cl | p-MeC₆H₄SF₅ |
| 23.5 hours | 1:0.46 | — |
| 29 hours | 1:0.39 | — |
| 48 hours | 1:0.024 | 41% |

From the comparison of the results of Example 21 with this Comparative Example 3, the reaction of p-methylphenylsulfur chlorotetrafluoride with the hydrous HF-pyridine at room temperature was remarkably faster than the reaction with the anhydrous HF-pyridine. The reaction with the hydrous HF-pyridine was completed in 24 hours, while the reaction of anhydrous HF-pyridine was not completed in 29 hours and a significant amount of the starting material still remained as seen from Table 10. In addition, the yield (65%) of the product obtained in 24 hours with the hydrous HF-pyridine was higher than the yield (41%) of the product obtained in 48 hours with anhydrous HF-pyridine.

### Example 22

### Synthesis of p-methylphenylsulfur pentafluoride from p-methylphenylsulfur chlorotetrafluoride with hydrous ZnF₂

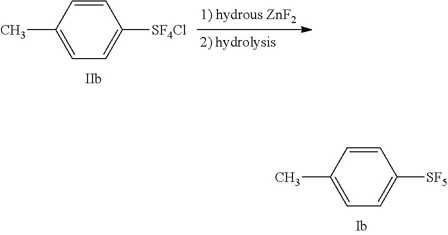

A reaction vessel made of fluoropolymer was charged with 287 mg (2.75 mmol) of ZnF₂ containing 1.5 wt % water and 1.0 g (4.26 mmol) of trans-p-methylphenylsulfur chlorotetrafluoride. The ZnF₂ containing 1.5 wt % water was prepared by adding the amount of water necessary for the content to anhydrous ZnF₂. The vessel was equipped with a condenser made of fluoropolymer and a balloon filled with N₂. The reaction mixture was stirred at 80° C. for 20.5 hours. At this time, the reaction was completed (p-methylphenylsulfur chlorotetrafluoride was consumed). An analysis of the reaction mixture by ¹⁹F-NMR showed that a mixture of p-methylphenylsulfur pentafluoride and p-methylphenylsulfonyl fluoride (as a byproduct) was produced at 83% and 1% yield, respectively.

The reaction mixture was extracted with diethyl ether and the ether solution (8 mL) was treated with 4 mL of 2.5 N aq. sodium hydroxide solution which contained benzyltrimethylammonium chloride (5 mol % to NaOH) (as a phase-transfer catalyst) at room temperature for 20 hours, leaving p-methylphenylsulfur pentafluoride as an only product quantitatively. All the p-methylphenylsulfonyl fluoride was hydrolyzed to p-methylphenylsulfonic acid salt and removed to the aqueous layer.

### Example 23

### Reaction of p-methylphenylsulfur chlorotetrafluoride with ZnF₂ hydrate

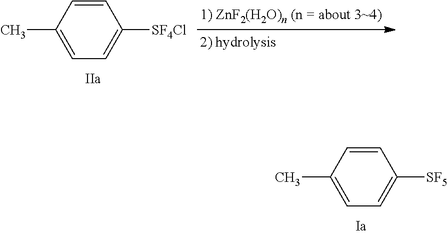

A reaction vessel made of fluoropolymer was charged with 2.0 g (8.53 mmol) of trans-p-methylphenylsulfur chlorotetrafluoride and 0.97 g of zinc difluoride hydrate, ZnF₂(H₂O)ₙ (n=about 3˜4) (from Sigma-Aldrich). The vessel was equipped with a condenser made of fluoropolymer and a balloon filled with N₂. The reaction mixture was stirred at 90° C. for 7 hours; at this point, the reaction was completed (p-methylphenylsulfur chlorotetrafluoride was consumed). An analysis of the reaction mixture by ¹⁹F-NMR showed that the reaction gave a 1:1.8 (mol ratio) mixture of p-methylphenylsulfur pentafluoride and p-methylphenylsulfonyl fluoride, and p-methylphenylsulfur pentafluoride and p-methylphenylsulfonyl fluoride were produced in 22% and 40% yields, respectively.

The reaction mixture was extracted with diethyl ether and the ether solution (about 20 mL) was treated with 13.6 mL of 2.5 N aq. sodium hydroxide solution which contained benzyltrimethylammonium chloride (5 mol % to NaOH) (as a phase-transfer catalyst) at room temperature for 6 hours, leaving p-methylphenylsulfur pentafluoride as an only product quantitatively. All the p-methylphenylsulfonyl fluoride was hydrolyzed to p-methylphenylsulfonic acid salt and removed to an aqueous layer.

### Example 24

### Reaction of p-methylphenylsulfur chlorotetrafluoride with 48 wt % hydrofluoric Acid

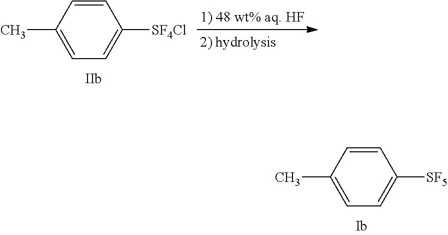

A reaction vessel made of fluoropolymer was charged with 1.0 g (4.26 mmol) of trans-p-methylphenylsulfur chlorotetrafluoride and 1.0 g of 48 wt % hydrofluoric acid (HF, 48 wt %; water, 52 wt %). The vessel was equipped with a condenser made of fluoropolymer and a balloon filled with N₂. The reaction mixture was stirred at 90° C. for 16.5 hours; at this point, the reaction was completed (p-methylphenylsulfur chlorotetrafluoride was consumed). An analysis of the reaction mixture by ¹⁹F-NMR showed that the reaction gave a 1:4 (mol ratio) mixture of p-methylphenylsulfur pentafluoride and p-methylphenylsulfonyl fluoride; p-methylphenylsulfur pentafluoride and p-methylphenylsulfonyl fluoride were produced in 11% and 44% yields, respectively.

The reaction mixture was neutralized with alkaline and extracted with diethyl ether. The ether solution (about 10 mL) was treated with 8.5 mL of 2.5 N aq. sodium hydroxide solution which contained benzyltrimethylammonium chloride (5 mol % to NaOH) (as a phase-transfer catalyst) at room temperature for 6 hours, leaving p-methylphenylsulfur pentafluoride as an only product quantitatively. All the p-methylphenylsulfonyl fluoride was hydrolyzed to p-methylphenylsulfonic acid salt and removed to an aqueous layer.

### Example 25

### Synthesis of p-fluorophenylsulfur pentafluoride from p-fluorophenylsulfur chlorotetrafluoride with hydrous hydrogen fluoride-pyridine

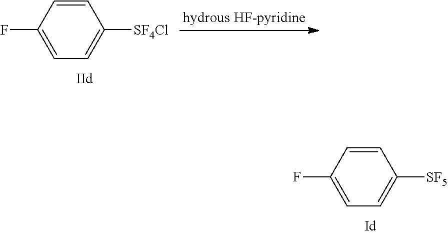

A reaction vessel made of fluoropolymer was charged with 1.02 g (4.26 mmol) of trans-p-fluorophenylsulfur chlorotetrafluoride and 0.55 mL of a mixture of 98.5 wt % hydrogen fluoride-pyridine (HF:pyridine=about 7:3 weight ratio) and 1.5 wt % water. A mixture of 98.5 wt % hydrogen fluoride-pyridine and 1.5 wt % water was prepared by adding the amount of water necessary for the content to a mixture of anhydrous hydrogen fluoride-pyridine (HF:pyridine=about 7:3 weight ratio) (from Sigma-Aldrich). The vessel was equipped with a balloon filled with N₂. The reaction mixture was stirred for 30 hours at 40° C. and then 21 hours at 50° C. An analysis of the reaction mixture by ¹⁹F-NMR showed that p-fluorophenylsulfur pentafluoride was produced in 56% yield and p-fluorophenylsulfonyl fluoride was produced in 5% yield as a byproduct.

Properties and spectral data of p-fluorophenylsulfur pentafluoride obtained are as follows; b.p. 71° C./80 mmHg; ¹H NMR (CDCl₃) δ 7.80-7.73 (m, 2H, aromatic), 7.17-7.09 (m, 2H, aromatic); ¹⁹F NMR (CDCl₃) δ 87.78-83.17 (m, 1F, SF), 63.81 (d, 4F, SF₄), −107.06 (s, 1F, CF); GC-MS m/z 222 (M⁺).

### Example 26

### Reaction of phenylsulfur chlorotetrafluoride and hydrous ZnF₂ Under a Flow of Chlorine (Presence of Halogen) at 100° C.

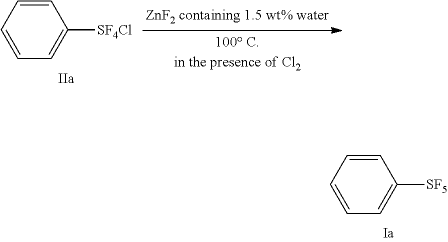

A reaction vessel made of fluoropolymer was charged with 5.0 g (22.7 mmol) of trans-phenylsulfur chlorotetrafluoride and 1.53 g of ZnF₂ (14.7 mmol) containing 1.5 wt % water. The ZnF₂ containing 1.5 wt % water was prepared by adding the amount of water necessary for the content to anhydrous ZnF₂. The reaction vessel was equipped with a condenser made of fluoropolymer and connected to a chlorine gas flowing device with a Cl₂ cylinder. The reaction vessel was filled with chlorine gas by flowing chlorine gas (10 mL/min, 19 minutes) into the vessel at room temperature. The reaction vessel was heated on an oil bath of 100° C. while chlorine gas was flown through the vessel at the flow rate of 5 mL/minute. The reaction mixture was analyzed in 5 hours by ¹⁹F NMR and the results are shown in Table 11.

TABLE 11
Reaction of phenylsulfur chlorotetrafluoride with
hydrous ZnF₂ at 100° C. in the presence of Cl₂

| Reaction | Molar ratio of | Yield of |
|---|---|---|
| time | PhSF₅:PhSF₄Cl | PhSF₅ |
| 5 hours | 1:0.05 | 75% |

As seen in Table 11, in 5 hours, the reaction gave a 1:0.05 mixture of phenylsulfur pentafluoride (PhSF₅): phenylsulfur chlorotetrafluoride (PhSF₄Cl, a mixture of trans- and cis-isomers). The reaction was close to completion and PhSF₅ was produced in 75% yield and ˜4% of PhSF₄Cl remained. As shown in Example 18, the reaction without the presence of chlorine gas gave a 1:0.9 mixture of PhSF₅:PhSF₄Cl in 5 hours. Thus, the comparison of Example 26 with Example 18 showed that the presence of halogen remarkably accelerate the reaction of phenylsulfur halotetrafluoride with a fluoride source under hydrous conditions. This is an impressive result as compared to other prior art methodologies.

The methods of the present invention, as described above, can be applied to the synthesis of other arylsulfur pentafluorides, properties and spectra data of which are shown in the following;

o-Fluorophenylsulfur pentafluoride (Ie); b.p. 91-94° C./120 mmHg; ¹H NMR (CDCl₃) δ 7.78-7.73 (m, 1H, aromatic), 7.55-7.48 (m, 1H, aromatic), 7.27-7.17 (m, 2H, aromatic); ¹⁹F NMR (CDCl₃) δ 82.38-81.00 (m, 1F, SF), 68.10 (dd, 4F, SF₄), −108.07-(−108.35) (m, 1F, CF).

p-Bromophenylsulfur pentafluoride (Ig); b.p. 77-78° C./10 mmHg; ¹H NMR (CDCl₃) δ 7.63 (s, 4H, aromatic); ¹⁹F NMR (CDCl₃) δ 84.13-82.53 (m, 1F, SF), 63.11 (d, 4F, SF₄).

m-Bromophenylsulfur pentafluoride (Ih); b.p. 69-70° C./10 mmHg; ¹H NMR (CDCl₃) δ 7.91 (t, 1H, aromatic), 7.72-7.64 (m, 2H, aromatic), 7.35 (t, 1H, aromatic); ¹⁹F NMR (CDCl₃) δ 83.55-82.47 (m, 1F, SF), 63.13 (d, 4F, SF₄).

p-Nitrophenylsulfur pentafluoride (Ii); b.p. 74-76° C./3 mmHg; ¹H NMR (CDCl₃) δ 8.36-8.30 (m, 2H, aromatic), 7.99-7.95 (m, 2H, aromatic); ¹⁹F NMR (CDCl₃) δ 82.32-80.69 (m, 1F, SF), 62.76 (d, 4F, SF₄).

2,6-Difluorophenylsulfur pentafluoride (Ij): m.p. 40.3-41.1 ° C.; ¹H NMR (CDCl₃) δ 7.51 (m, 1H), 7.04 (m, 2H); ¹⁹F NMR (CDCl₃) δ 82.32-80.69 (m, 1F, SF), 62.76 (d, 4F, SF₄); high resolution mass spectrum; found 239.984509 (calcd for C₆H₃F₇S; 239.984370); elemental analysis, calcd for C₆H₃F₇S; C, 30.01%, H, 1.26%; found, C, 30.20%, H, 1.47%.

3-Chloro-2,6-difluorophenylsulfur pentafluoride (Ik): ¹H NMR (CDCl₃) δ 7.60 (m, 1H), 7.04 (m, 1H); ¹⁹F NMR (CDCl₃) δ 77.9-75.7 (m, 1F, SF), 73.2-72.5 (m, 4F, SF₄), −103.3 (m, 1F), −105.2 (m, 1F); high resolution mass spectrum, found 275.942071 (36.0%) (calcd for C₆H₂³⁷ClF₇S; 275.942447), found 273.945943 (100%) (calcd for C₆H₂³⁵ClF₇S; 273.945397).

2,4,6-Trifluorophenylsulfur pentafluoride (Il): ¹H NMR (CDCl₃) δ 6.80 (t, J=8.6 Hz, 3.5-H); ¹⁹F NMR (CDCl₃) δ 78.7-75.3 (m, SF), 73.8-72.9 (m, SF₄), −100.6 (m, 4-F), −100.7 (m, 2.6-F); GC-Mass m/z 258 (M⁺).

3-Chloro-2,4,6-trifluorophenylsulfur pentafluoride (Im): ¹H NMR (CDCl₃) δ 6.95 (br.t, J=9.5 Hz, 5-H); ¹⁹F NMR (CDCl₃) δ 78.7-75.3 (m, SF), 73.8-72.9 (m, SF₄), −101.3 (m, 2 or 6-F), −102.3 (m, 4-F), −102.6 (m, 2 or 6-F); GC-Mass m/z 294, 292 (M⁺).

2,3,4,5,6-Pentafluorophenylsulfur pentafluoride (In): b.p. 135-137° C.; ¹⁹F NMR (CDCl₃) δ 74.8 (m, 5F, SF₅), −133.4 (m, 2F, 2,6-F), −146.2 (m, 1F, 4-F), −158.6 (m, 2G, 3.5-F); ¹³C NMR (CDCl₃) δ 143.6 (dm, J=262.2 Hz), 137.9 (dm, J=253.6 Hz), 126.7 (m). High resolution mass spectrum; found 293.956492 (calcd for C₆F₁₀S; 293.956104).

### Example 27

### Reactions of phenylsulfur chlorotetrafluoride and ZnF₂ Under No Flow, Slow Flow, and Fast Flow of an Inactive Gas (Nitrogen)

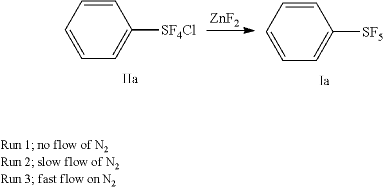

Run 1 with a Balloon Filled with N₂ (No Flow of N2k

In a dry box, a reaction vessel made of fluoropolymer was charged with 1.0 g (4.54 mmol) of trans-phenylsulfur chlorotetrafluoride (trans-PhSF₄Cl) and 0.28 g (2.7 mmol) of anhydrous ZnF₂. The reaction vessel was removed from the dry box, and equipped with a condenser made of fluoropolymer and a balloon filled with N₂. The reaction mixture was stirred at 120° C. for 4 hours. The reaction mixture was analyzed by ¹⁹F NMR, results are shown in Table 12.

Run 2 with N₂ Flow of a Rate of 5.4 mL/min:

In a dry box, a 50 mL reaction vessel made of fluoropolymer was charged with 10.0 g (0.045 mol) of trans-PhSF₄Cl and 2.8 g (0.027 mol) of anhydrous ZnF₂. The reaction vessel was removed from the dry box, and equipped with a condenser made of fluoropolymer and connected to a N₂ gas flowing device. The reaction mixture was slowly heated to 120° C. with N₂ flowing at the rate of 5.4 mL/minute. The reaction mixture was stirred at 120° C. with N₂ flowing for 5 hours. After being cooled to room temperature, the reaction mixture was analyzed with ¹⁹F NMR. The results are shown in Table 12.

Run 3 with N₂ Flow of a Rate of 26.9 mL/min:

In a dry box, a 50 mL reaction vessel made of fluoropolymer was charged with 10.0 g (0.045 mol) of trans-PhSF₄Cl and 2.8 g (0.027 mol) of anhydrous ZnF₂. The reaction vessel was removed from the dry box, and equipped with a condenser made of fluoropolymer and connected to a N₂ gas flowing device. The reaction mixture was slowly heated to 120° C. with N₂ flowing at a rate of 26.9 mL/minute. The reaction mixture was stirred at 120° C. with N₂ flowing for 5 hours. After being cooled to room temperature, the reaction mixture was analyzed with ¹⁹F NMR. The results are shown in Table 12.

TABLE 12
Reactions of phenylsulfur chlorotetrafluoride with
ZnF₂ under no flow and slow and fast flow of N₂

| | | Reaction | Molar ratio of | Yield of |
|---|---|---|---|---|
| Run | N₂ flow | time | PhSF₅:PhSF₄Cl* | PhSF₅ |
| Run 1 | no flow | 4 hours | PhSF₄Cl was consumed | 88% |
| Run 2 | 5.4 mL/min | 5 hours | 1:0.35 | 67% |
| Run 3 | 26.9 mL/min | 5 hours | 1:1.39 | 38% |

*PhSF₅ = phenylsulfur pentafluoride. PhSF₄Cl; a mixture of trans- and cis-isomers.

The comparison of Run 1 and Run 2 showed that the reaction under the flow of nitrogen was slowed down. The comparison of Run 2 and Run 3 showed that the reaction under the fast flow of nitrogen was slowed down more than the reaction under the slow flow of nitrogen. Thus, a flow of inactive gas has an inhibitory effect on reaction rate and yields.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

1. A method for preparing an arylsulfur pentafluoride having a formula (I) as follows: 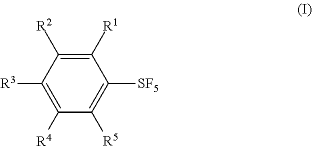
the method comprising: reacting arylsulfur halotetrafluoride having a formula (II) with a fluoride source under hydrous conditions; 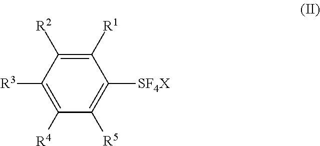
wherein R¹, R², R³, R⁴, and R⁵ each is independently a hydrogen atom, a halogen atom, a substituted or unsubstituted alkyl group having 1 to 18 carbon atoms, a substituted or unsubstituted aryl group having 6 to 30 carbon atoms, a nitro group, a cyano group, a substituted or unsubstituted alkanesulfonyl group having 1 to 18 carbon atoms, a substituted or unsubstituted arenesulfonyl group having 6 to 30 carbon atoms, a substituted or unsubstituted alkoxy group having 1 to 18 carbon atoms, a substituted or unsubstituted aryloxy group having 6 to 30 carbon atoms, a substituted or unsubstituted acyloxy group having 1 to 18 carbon atoms, a substituted or unsubstituted alkanesulfonyloxy group having 1 to 18 carbon atoms, a substituted or unsubstituted arenesulfonyloxy group having 6 to 30 carbon atoms, a substituted or unsubstituted alkoxycarbonyl group having 2 to 18 carbon atoms, a substituted or unsubstituted aryloxycarbonyl group having 7 to 30 carbon atoms, a substituted carbamoyl group having 2 to 18 carbon atoms, a substituted amino group having 1 to 18 carbon atoms, or a SF₅ group; and X is a chlorine atom, a bromine atom, or an iodine atom.

2. The method of claim 1 wherein the hydrous conditions are established at least by using a non-anhydrous fluoride source.

3. The method of claim 1 wherein the fluoride source is at least one member selected from a group consisting of fluorides of typical elements in the Periodic Table, fluorides of transition elements in the Periodic Table, and mixtures or compounds between or among these fluorides of typical elements and/or transition elements, as well as mixtures, salts, or complexes of these fluorides with organic molecules.

4. The method of claim 3, wherein the fluorides of typical elements in the Periodic Table are hydrogen fluoride and fluorides of the Elements 13-15 and wherein the fluorides of transition elements in the Periodic Table are fluorides of Elements 11 and 12.

5. The method of claim 1 wherein X is a chlorine atom.

6. The method of claim 1, further comprising the reaction of an arylsulfur halotetrafluoride with a fluoride source under hydrous conditions wherein the reaction is conducted in the presence of a halogen selected from the group of chlorine, bromine, iodine, and interhalogens.

7. A method for preparing an arylsulfur pentafluoride having a formula (I) as follows: 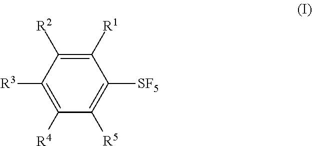
the method comprising reacting arylsulfur halotetrafluoride having a formula (II) with a fluoride source under hydrous conditions, and treating the resulting reaction products with hydrolysis conditions; 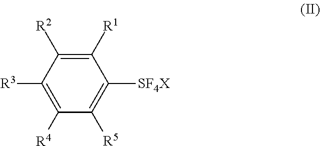
wherein R¹, R², R³, R⁴, and R⁵ each is independently a hydrogen atom, a halogen atom, a substituted or unsubstituted alkyl group having 1 to 18 carbon atoms, a substituted or unsubstituted aryl group having 6 to 30 carbon atoms, a nitro group, a cyano group, a substituted or unsubstituted alkanesulfonyl group having 1 to 18 carbon atoms, a substituted or unsubstituted arenesulfonyl group having 6 to 30 carbon atoms, a substituted or unsubstituted alkoxy group having 1 to 18 carbon atoms, a substituted or unsubstituted aryloxy group having 6 to 30 carbon atoms, a substituted or unsubstituted acyloxy group having 1 to 18 carbon atoms, a substituted or unsubstituted alkanesulfonyloxy group having 1 to 18 carbon atoms, a substituted or unsubstituted arenesulfonyloxy group having 6 to 30 carbon atoms, a substituted or unsubstituted alkoxycarbonyl group having 2 to 18 carbon atoms, a substituted or unsubstituted aryloxycarbonyl group having 7 to 30 carbon atoms, a substituted carbamoyl group having 2 to 18 carbon atoms, a substituted amino group having 1 to 18 carbon atoms, or a SF₅ group; and X is a chlorine atom, a bromine atom, or an iodine atom.

8. The method of claim 7 wherein the hydrous conditions are established at least by using a non-anhydrous fluoride source.

9. The method of claim 7 wherein the fluoride source is at least one member selected from a group consisting of fluorides of typical elements in the Periodic Table, fluorides of transition elements in the Periodic Table, and mixtures or compounds between or among these fluorides of typical elements and/or transition elements, as well as mixtures, salts, or complexes of these fluorides with organic molecules.

10. The method of claim 7, wherein the fluorides of typical elements in the Periodic Table are hydrogen fluoride and fluorides of the Elements 13-15 and wherein the fluorides of transition elements in the Periodic Table are fluorides of Elements 11 and 12.

11. The method of claim 7 wherein X is a chlorine atom.

12. The method of claim 7, further comprising the reaction of an arylsulfur halotetrafluoride with a fluoride source under hydrous conditions wherein the reaction is conducted in the presence of a halogen selected from the group of chlorine, bromine, iodine, and interhalogens.

13. The method for purifying an arylsulfur pentafluoride having formula (I) as follows: 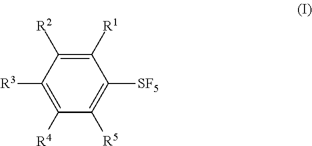
the method comprising treating a mixture containing the arylsulfur pentafluoride with hydrolysis conditions;
wherein R¹, R², R³, R⁴, and R⁵ each is independently a hydrogen atom, a halogen atom, a substituted or unsubstituted alkyl group having 1 to 18 carbon atoms, a substituted or unsubstituted aryl group having 6 to 30 carbon atoms, a nitro group, a cyano group, a substituted or unsubstituted alkanesulfonyl group having 1 to 18 carbon atoms, a substituted or unsubstituted arenesulfonyl group having 6 to 30 carbon atoms, a substituted or unsubstituted alkoxy group having 1 to 18 carbon atoms, a substituted or unsubstituted aryloxy group having 6 to 30 carbon atoms, a substituted or unsubstituted acyloxy group having 1 to 18 carbon atoms, a substituted or unsubstituted alkanesulfonyloxy group having 1 to 18 carbon atoms, a substituted or unsubstituted arenesulfonyloxy group having 6 to 30 carbon atoms, a substituted or unsubstituted alkoxycarbonyl group having 2 to 18 carbon atoms, a substituted or unsubstituted aryloxycarbonyl group having 7 to 30 carbon atoms, a substituted carbamoyl group having 2 to 18 carbon atoms, a substituted amino group having 1 to 18 carbon atoms, or a SF₅ group.
